(19) United States
(12) Patent Application Publication
(10) Pub. No.: US 2002/0082668 A1
(43) Pub. Date: Jun. 27, 2002

(54) Skin-gripper

(76) Inventor: Dov Ingman, Haifa (IL)

Correspondence Address: William H. Dippert, Esq., Cowan, Liebowitz & Latman, P.C., 1133 Avenue of the Americas, New York, NY, 10036-6799, US

(21) Appl. No.: 10/053,393

(22) Filed: Nov. 7, 2001

Related U.S. Application Data
(63) Continuation of application No. PCT/IL00/00714, filed on Nov. 2, 2000.

(30) Foreign Application Priority Data
Nov. 2, 1999 (IL) PCT/IL99/00584

(51) Int. Cl. A61F 7/00

(52) U.S. Cl. 607/98; 601/2; 600/9

(57) ABSTRACT

A method for treating blemishes having furrows on a region of a person's skin comprising: positioning a surface of a bracing body so that the surface contacts or is proximate to the region skin; and attracting the region of skin to the surface so that blemishes in the region of skin are flattened to the surface.

### RELATED APPLICATIONS

The present application is a continuation of PCT/IL00/00714 designating the U.S. Pat. No., filed Nov. 2, 2000, which claims priority from PCT/IL99/00584, filed Nov. 2, 1999, the disclosures of which are incorporated herein by reference.

### FIELD OF THE INVENTION

The invention relates to apparatus and methods for attracting and holding fast areas of skin using electric fields and for using such apparatus and methods for therapeutic and cosmetic treatment of the skin and for attaching jewelry, clothes and other objects to the skin.

### BACKGROUND OF THE INVENTION

There are very few methods for grasping and holding the skin or for attaching and fixing objects and decorations directly to the skin. Skin is generally grasped and held by clamping or pinching it. Objects to be attached to the skin are either tied or strapped to a body part or attached to the skin with a glue, adhesive or paste. In some cases the skin is pierced to attach an object, such as an earring, to the skin.

It would be advantageous to have a method of attaching bandages, sensors, therapeutic devices, and decorative and protective objects to the skin by simply placing them in contact with the skin without the need to pierce or deform the skin or use a glue, adhesive or gel.

### SUMMARY OF THE INVENTION

An aspect of some embodiments of the present invention relates to providing an apparatus, hereinafter referred to as a “skin-gripper”, that comprises a surface, which when placed in contact with a region of a person's skin generates strong forces that hold the region to the surface. The surface of a skin-gripper that attracts and holds the skin is hereinafter referred to as a “gripping surface”.

In an embodiment of the present invention, a skin-gripper comprises a conducting layer, which comprises at least one conductor having a surface covered with a thin insulating dielectric layer, and an electric power supply for electrifying the conductor. The power supply is grounded to the skin so that when the power supply electrifies the at least one conductor, a potential difference is generated between the at least one conductor and the skin. Preferably, the conducting layer is formed or bonded to an appropriate insulating substrate. The dielectric layer is formed from a material that has a relatively high dielectric constant and high breakdown voltage.

When the conductor is held so that the dielectric layer is contiguous with a region of the skin, the power supply is operated to provide a potential difference between the skin and the conductor. The skin, because of electrolytes in the skin and in tissue below the skin, acts as a conductor. Therefore, as a result of the potential difference, substantially equal and opposite surface charge densities are generated on the surface of the at least one conductor and the region of skin to which the conductor is pressed. The region of skin is thereby strongly attracted to and held fast to the dielectric layer on the conductor surface. The surface of the dielectric layer to which the skin is held fast is a gripping surface of the skin-gripper.

The at least one conductor, the skin to which it is attracted and the thin dielectric insulating layer between them operate as a capacitor having a dielectric layer between the capacitor's plates. The force that attracts and holds the skin to the conductor may therefore be estimated from the usual capacitor formulae. For appropriate choices of potential difference between the conducting layer and skin, thickness and dielectric constant of the insulating layer, in accordance with an embodiment of the present invention, the force that attracts the region of skin to the surface of the electrode attains values between about 10-100 Newtons per cm² of conductor surface. This attractive force has the same effect in holding the skin and conductor together as if the skin and conductor were pressed together with a pressure of between about 1-10 atmospheres.

In order for the attractive force to attain the magnitudes noted above, the surface of the dielectric, i.e. the gripping surface, must be in close contact with the skin to which it is pressed. In some cases, body hair may interfere with the required close contact between a gripping surface of a skin-gripper and skin to which the skin-gripper is placed in contact. To assure appropriate close contact, in some embodiments of the present invention, the dielectric layer of the skin-gripper is formed sufficiently flexible so that the gripping surface bends, or “molds itself”, around body hair and contacts regions of bare skin between body hair follicles. For some applications, to assure appropriate contact, body hair on a region of skin is shaved before the gripping surface of a skin-gripper is placed in contact with region of skin.

In accordance with some embodiments of the present invention a skin-gripper is formed so that its gripping surface is in the shape of a comb that has a plurality of coplanar flexible closely spaced like teeth. To place the skin-gripper in close contact with the skin, the plane of the comb like teeth is placed flush with the skin, i.e. with the teeth parallel to the skin, and “threaded” between the body hair.

An aspect of some embodiments of the present invention relates to providing a bandage, hereinafter referred to as an “electro-patch bandage”, for protecting skin wounds that is held in place over the wound by a skin-gripper. The electro-patch bandage does not require adhesive to remain in place over the wound. In addition it provides an electrostatic field that is beneficial to the healing process of the wound.

An electro-patch bandage, in accordance with an embodiment of the present invention, optionally comprises a flexible skin-gripper comprising a flexible insulating substrate to which a conducting layer having at least one thin pliable conductor is bonded. A flexible layer of dielectric material covers the conducting layer to form the gripping surface. An appropriate power supply electrifies the at least one conductor in the conducting layer.

When the electro-patch bandage is placed to cover a wound on a person's skin, the power supply electrifies the at least one conductor to generate a potential difference, hereinafter referred to as a “gripping voltage” between the at least one conductor and the skin. The magnitude of the gripping voltage is such that the bandage is firmly held in place over the wound by the force with which the gripping surface attracts the skin. Furthermore, as a result of the force of attraction and the flexibility of the gripping surface, the electro-patch bandage conforms itself to the shape of the body in the region of the wound and seals the region of the wound against ingress of contaminants from the environment.

In some electro-patch bandages, in accordance with some embodiments of the present invention the dielectric is designed to break down at appropriate gripping voltages to enable small micro-currents of electricity to flow between the conducting layer of the skin-gripper and the skin in contact with the bandages gripping surface. The micro-currents are beneficial for skin health and have an analgesic effect.

In some embodiments of the present invention an electro-patch bandage comprises a heating element that heats the region of skin in contact with the electro-patch bandage. In accordance with some embodiments of the present invention an electro-patch bandage incorporates ultrasound transducers for radiating acoustic waves into tissue in the region of a wound protected by the bandage.

In some embodiments of the present invention, the electro-patch bandage is perforated with a plurality of micro-holes formed in the material of the bandage using methods known in the art so that a wound and skin covered with the electro-patch bandage “can breathe”. Preferably, inside surfaces of the micro-holes are coated with a thin layer of insulating material. The insulating material completely covers the walls of the holes and is bonded to both the material of the dielectric layer and the material of the insulating substrate of the electro-patch bandage. This prevents sweat from short circuiting the conducting layer of the electro-patch bandage to skin to which the electro-patch bandage is applied. The insulating layer may be applied to the inside surfaces of the micro-holes using various methods known in the art. For example, the insulator can be applied to the surfaces of the micro-holes in a dipping or spraying process that is used to form the dielectric layer and/or the insulating substrate

In some embodiments of the present invention the power supply is small and light enough to be mounted to or integrated with the layers forming the gripping surface. In other embodiments of the present invention the power supply is separate from the layers and electrically connected to the at least one conductor in the conducting layer using appropriate conducting wires.

According to an aspect of some embodiments of the present invention, the at least one conductor comprises a plurality of conductors. The conductors are optionally arranged in an array on the insulating substrate and electrified to provide an electric field having substantial components parallel to the surface of the skin to which the electro-patch bandage is applied. Electric fields parallel to the skin tend to promote migration of epidermal cells, which promotes closing of wounds. To provide such a field, in accordance with an embodiment of the present invention, adjacent conductors in the array are charged to opposite polarity by the power supply.

According to an aspect of some embodiments of the present invention a skin-gripper, in accordance with an embodiment of the present invention, is used to provide a device for perfusing a medication or other appropriate substance, hereinafter referred to generically as a “medication”, into the body through the skin. The medication, in the form of a paste or gel, is preferably applied in a thin layer to the gripping surface of the skin-gripper. When the skin-gripper is placed on the skin and “electrified”, the medication is squeezed between the skin and the gripping surface and perfused through the skin into the body by the force with which the skin-gripper attracts the skin. If the medication comprises charged particles, such as charged colloidal particles or molecules to be delivered to the body, perfusion is enhanced by electrophoresis resulting from the electric field between the gripping surface and the skin. The rate at which the medication perfuses into the body is controlled by the area of the gripping surface, the magnitude of the force of attraction between the gripping surface and the skin and the length of time that the skin-gripper is in place on the skin.

According to an aspect of some embodiments of the present invention, a skin-gripper used to perfuse a medication transdermally through a region of skin comprises a sensor for sensing electrical fields in the skin and underlying tissue. The sensor transmits signals responsive to the electrical fields to a controller that processes the signals to determine frequencies of peristalsis in pores of the skin. The controller modulates voltage applied between the skin-gripper and the skin responsive to the identified peristaltic frequencies. In some embodiments of the present invention, the controller modulates the applied voltage at a frequency that is substantially equal to the “peristaltic frequency”. Electrical stimulation of the skin at a peristaltic frequency tends to aid transdermal transmission of a medication being perfused by a skin-gripper, in accordance with an embodiment of the present invention.

An aspect of some embodiments of the present invention relates to providing apparatus, hereinafter referred to as a “massager”, that comprises a skin-gripper for therapeutic mechanical manipulation and massaging of a person's skin.

According to an embodiment of the present invention, a massager comprises a skin-gripper having a shaped gripping surface suitable for massaging the skin. To massage a person's skin, an operator of the massager electrifies the skin-gripper and holds it so that its gripping surface attaches firmly to a region of skin. The operator manipulates the skin-gripper to stretch, pull and massage the region of skin and underlying tissue. Various different shapes for the gripping surface, in accordance with embodiments of the present invention, are possible and advantageous. For example, the gripping surface may be planar or curved or have a corrugated shape.

In some massagers, in accordance with embodiments of the present invention, the dielectric is designed to break down at appropriate gripping voltages to enable small micro-currents of electricity to flow between the conducting layer of the skin-gripper and the skin in contact with the gripping surface.

An aspect of some embodiments of the present invention relates to providing a massager, hereinafter referred to as a “stretch massager”, comprising a skin-gripper that has an elastically stretchable gripping surface.

In some embodiments of the present invention, the skin-gripper is formed from a conducting layer optionally having a dense array of small thin conductors that are laminated or otherwise bonded to an elastically stretchable insulating substrate using methods known in the art. A thin elastically stretchable dielectric layer having appropriate dielectric constant and/or breakdown voltage covers the conducting layer. A power supply is electrically connected to each conductor using methods known in the art so that when the skin-gripper is stretched, proper electrical contact is maintained between the power supply and each of the conductors in the array.

Stretch massagers are used, in accordance with some embodiments of the present invention, for exercising and stimulating skin in order to tone the skin and ameliorate wrinkles. To operate a stretch massager, in accordance with an embodiment of the present invention, the gripping surface of the stretch massager is positioned on a person's skin and electrified to an appropriate gripping voltage. Areas of skin on edges of wrinkles in the skin contact the gripping surface and are held fast to it by forces of attraction between the skin and the gripping surface. Areas of skin in furrows between edges of wrinkles are displaced from the gripping surface and are not held by the gripping surface. However, when the gripping surface is stretched, edges of wrinkles held by the gripping surface are pulled away from each other. Wrinkles are flattened out and skin areas in furrows of the flattened wrinkles come into contact with the gripping surface and are thereafter held fast to the gripping surface. Repeated cycling of the gripping surface between stretched and non-stretched states massages the skin and is beneficial for the removal of wrinkles and for improving skin tone.

Whereas stretch massagers, in accordance with embodiments of the present invention are used to flatten and “iron” out wrinkles, other massagers, in accordance with embodiments of the present invention are also used to ameliorate wrinkling. For example, by pressing a gripping surface of a massager, in accordance with an embodiment of the present invention, to a wrinkled area of skin with sufficient pressure, some wrinkles will be pressed flat and held flat to the gripping surface. When the massager is moved to massage the area of skin, the massaging and flattening of the wrinkles is beneficial for the removal of the wrinkles.

An aspect of some embodiments of the present invention relates to providing a method and apparatus that use magnetic fields for treating and ameliorating wrinkles.

In an embodiment of the present invention, a thin pliable magnetized foil is stuck, using an appropriate adhesive known in the art, to a region of skin having wrinkles. The foil is “kneaded” to the skin. The thickness of the foil and its Young's modulus are determined so that the pliability of the foil is such that when the foil is kneaded to the skin, the foil molds to wrinkles in the skin and sticks to skin in furrows of the wrinkles. After the foil is stuck and kneaded to the skin, a relatively thick “bracing” layer of, optionally pliable, magnetic material is pressed to the foil. In embodiments in which the bracing layer is pliable, the bracing layer is substantially less pliable than the thin foil and does not mold itself to furrows of wrinkles on the skin.

Attractive magnetic forces between the foil and the bracing layer flatten the foil and the wrinkles to the bracing layer. The bracing layer and the foil are left in place for a convenient period of time, such as the duration of an afternoon nap or of a mudpack application in a beauty parlor. The treatment is repeated periodically. In some embodiments of the present invention, the bracing layer is formed so that a surface of the bracing layer that is pressed to the wrinkle foil matches contours of the body part covered by the region of skin being treated.

In some embodiments of the present invention, vacuum is used in addition to or instead of magnetic forces to flatten a foil, hereinafter referred to as a “wrinkle foil”, used to treat wrinkles to a bracing layer. In some embodiments of the present invention, electrostatic forces are used to adhere a wrinkle foil to furrows of wrinkles in a region of skin being treated to reduce skin wrinkles.

According to an aspect of some embodiments of the present invention in which a bracing layer and/or wrinkle foil is used to treat wrinkles in a region of skin, a “perfusion skin-gripper” is used to perfuse a substance beneficial for the treatment of wrinkles transdermally. The vacuum and/or electrical and/or mechanical stimulation of the treated region augments metabolism in the region and tends to draw the transdermally introduced substance to the treated area. In some embodiments of the present invention, the treated region of skin and the region of skin through which the substance is perfused are electrically stimulated responsive to frequencies of peristalsis of pores in the regions to augment transdermal perfusion and transport of the substance.

Whereas apparatus and methods in accordance with embodiments of the present invention have been described as being used for, among other applications, treating wrinkles, it should be noted that such apparatus and methods are usable for treating “mechanical” skin blemishes other than wrinkles. They may be used, for example, to treat and reduce scarring from wounds and acne.

An aspect of some embodiments of the present invention relates to providing a massager, hereinafter referred to as a “pattern massager” that massages a region of a patient's skin by applying attraction forces to the skin in different temporal and spatial patterns.

In some embodiments of the present invention, a pattern massager comprises a skin-gripper that has a flexible gripping surface provided with a plurality of conductors that are electrified independently of each other by an appropriate power supply and switching circuitry. To massage a region of a patient's skin, the gripping surface is placed in contact with the region of skin and voltage differences are applied between conductors in the gripping surface and the skin in different spatial and temporal patterns. The different temporal and spatial voltage patterns generate different patterns of attractive forces between the gripping surface and the skin that travel over the skin to massage and manipulate the skin.

An aspect of some embodiments of the present invention relates to providing massagers, hereinafter referred to as “motile massagers” that are capable of independently moving over a person's skin.

According to some embodiments of the present invention a motile massager comprises a skin-gripper having a circularly cylindrical gripping surface. In accordance with some embodiments of the present invention, the gripping surface is formed by adhering or forming a dense array of narrow rectangular strip conductors on a cylindrical insulating substrate surface. The strip conductors are parallel to each other and to the axis of the substrate cylinder and optionally, run the length of the cylinder. Optionally, all the strip conductors have a same width. An appropriate dielectric layer covers the strip conductors. The motile massager comprises a power supply and switching circuit that can preferably electrify each of the strip conductors independently of each other.

When the motile massager is placed on a person's skin only a relatively narrow region of its cylindrical gripping surface (i.e. a strip of its surface parallel to the axis of the gripping surface) contacts the skin. At any one time, only a relatively small group of strip conductors that are located opposite the region of skin that is in contact with the gripping surface are electrified by the power supply to grip the skin and hold the motile massager to the skin. The group of electrified strip conductors is hereinafter referred to as a “gripping group”. The power supply and switching circuit sequentially electrify different strip conductors to shift the position of the gripping group and thereby cause the gripping body to roll over the skin. As the motile massager rolls along the skin it exerts attractive forces on the skin that massage the skin and increase blood flow to the skin.

To understand how the gripping surface is made to roll, let the extreme electrified strip conductors that bound a gripping group be referred to as a first and last strip conductor. If the last strip conductor of a gripping group is grounded and the non-electrified strip conductor adjacent to the first strip conductor electrified, then the gripping surface will roll a short distance in the direction of the first electrode. If the power supply and switching circuit are controlled to repeatedly ground the last electrode and electrify the non-electrified strip electrode adjacent to the first electrode of a gripping group, the motile massager will roll over the person's skin in the direction of the first electrode.

Other geometries for motile massagers that stick to and roll or crawl along the skin, in accordance with an embodiment of the present invention, are possible and will occur to persons of the art. For example a cylindrical gripping surface might be defined by a directrix that is an equilateral polygon or a motile massager might comprise more than one cylindrical gripping surface.

An aspect of some embodiments of the present invention relates to providing a massager, in accordance with embodiments of the present invention that incorporates elements that provide skin treatment modalities additional to the massaging functions of the massager.

For example, in an embodiment of the present invention a massager comprises heating elements that heat a region of skin being massaged by the massager. In accordance with another embodiment of the present invention a massager incorporates ultrasound transducers for radiating acoustic waves into skin being massaged and tissue below the skin. The acoustic waves are useful for pain relief and aid in the breakup and absorption of salt deposits that form in joints and articulations and which often result in debility.

An aspect of some embodiments of the present invention relates to providing a massager, hereinafter referred to as a “vacuum massager”, for treating a person's skin in which a skin-gripper is connected to a vacuum pump. In some embodiments of the present invention, the layers that determine the shape of the gripping surface are flexible so that the gripping surface of the skin-gripper is flexible. The vacuum pump removes air and provides a partial vacuum between the gripping surface of the skin-gripper and a region of skin to which the skin-gripper is applied. The vacuum massager, as with other massagers in accordance with embodiments of the present invention, may comprise heating elements and/or ultrasound transducers. The action of the vacuum, in conjunction with electric fields in the skin and tissue under the skin generated by voltages applied to conductors in the conducting layer of the skin 20 gripper, and/or ultrasound waves, and/or heat, aids in the removal of oils and pollutants from the skin and tissue below the skin.

An aspect of some embodiments of the present invention relates to providing ornaments, clothes items and protective wear that are attached to a person's skin using at least one skin-gripper, in accordance with an embodiment of the present invention.

In an embodiment of the present invention a decorative element such as for example a decal, small “light show” display or a piece of jewelry is attached to a thin skin-gripper. A person wears the ornament by simply pressing the skin-gripper to an appropriate area of his or her skin.

Clothes items and protective wear can similarly be attached to a person's skin using skin-grippers. A nose guard used to protect a person's nose from sunlight can easily be kept in place using a skin-gripper, in accordance with an embodiment of the present invention. Earplugs used by swimmer's to prevent water from entering their ears are notoriously difficult to keep properly in place.

An aspect of some embodiments of the present invention relates to providing wearable decorative items that move over a person's body. For example, a small motile massager (hereinafter referred to as a “motile skin-gripper” when not used as a massager) might be disguised as a beautiful caterpillar that slowly creeps along the bared shoulders of a woman in a topless evening gown. Or a motile skin-gripper might be used to provide a choker that revolves slowly around the neck of its wearer to display different decorative elements in the choker.

There is therefore provided in accordance with an embodiment of the present invention, a method for treating blemishes having firrows on a region of a person's skin comprising: positioning a surface of a bracing body so that the surface contacts or is proximate to the region skin; and attracting the region of skin to the surface so that blemishes in the region of skin are flattened to the surface.

In some embodiments of the present invention attracting the region of skin comprises adhering a thin foil to the skin and attracting the thin foil to the surface of the bracing body. Optionally, adhering comprises kneading the foil so that it contacts and sticks to skin in furrows of blemishes in the region of skin. Optionally, adhering comprises forcing air between the surface of the bracing body and the foil so as to increase pressure between the bracing body surface and the foil and press thereby the foil into furrows of the blemishes.

In some embodiments of the present invention, adhering to the skin comprises adhering with an adhesive. In some embodiments of the present invention, adhering to the skin comprises charging the foil with respect to the skin so that electrostatic forces between the charged foil and induced charge in the skin adhere the foil to the skin.

In some embodiments of the present invention the foil is a magnetized foil. In some embodiments of the present invention attracting comprises positioning a magnetized element in proximity to the magnetized foil so that magnetic forces between the element and the foil attract the foil to the surface of the bracing body. Optionally, the bracing body comprises the magnetized element.

In some embodiments of the present invention the bracing body surface is a surface of a thin layer of dielectric material bonded to a conductor and attracting the skin comprises applying a voltage between the conductor and the skin. Optionally, attracting the region of skin comprises pressing the dielectric surface of the skin-gripping apparatus to the region of skin so that furrows of blemishes in the skin are flattened to and contact the dielectric surface.

In some embodiments of the present invention attracting the region of skin comprises aspirating air from between the region of skin and the surface of the bracing body.

In some embodiments of the present invention the method comprises shaping the surface of the bracing body so that the contour of the surface is substantially a negative of the contour of the region of skin exclusive of contour features resulting from the blemishes in the skin.

There is further provided in accordance with an embodiment of the present invention, a method for treating blemishes having firrows on a region of a person's skin comprising: placing on the region of skin a thin pliable foil that at least partially recovers its shape after being deformed when forces responsible for the deformation are removed; applying forces that deform the foil so that it contacts skin in furrows of the blemish; adhering the deformed foil to the blemish; and removing the applied forces so that the foil at least partially recovers its undeformed shape.

Optionally, adhering comprises using an adhesive. Optionally, adhering comprises charging the foil with respect to the skin so that electrostatic forces between the charged foil and induced charge in the skin adhere the foil to the skin.

In some embodiments of the present invention the method comprises heating the region of skin. In some embodiments of the present invention the method comprises cooling the region of skin.

In some embodiments of the present invention the method comprises: heating the skin while the blemishes are flattened to the surface to soften collagen fibers in the skin; and subsequently cooling the skin while the blemishes remain flattened to the surface so that the collagen fibers retain a memory of their flattened configuration.

Optionally, the method comprises mechanically stimulating the region of skin using ultrasound waves. Optionally, mechanically stimulating comprises stimulating the skin at a resonant frequency of vibration of the skin. Optionally, mechanically stimulating comprises determining a resonant frequency of vibration of the skin using ultrasound.

In some embodiments of the present invention the method comprises stimulating the region of skin at a frequency of peristaltic waves of pores in the skin. Optionally, stimulating the region of skin at the peristaltic frequency of the pores comprises stimulating the skin electrically. Additionally or alternatively, stimulating the region of skin at the peristaltic frequency comprises stimulating the skin mechanically.

In some embodiments of the present invention the method comprises perfusing a substance beneficial for treating the blemish transdermally. Optionally, perfusing comprises: sandwiching the substance between a region of skin and a surface of a conductor coated with a thin dielectric layer; and applying a voltage between the conductor and the skin. The method, optionally, comprises controlling the voltage to control the rate at which the substance is perfused. Optionally the method comprises modulating the voltage at a frequency of peristaltic waves of pores in the region of skin.

In some embodiments of the present invention, a blemish comprises a wrinkle.

There is further provided in accordance with an embodiment of the present invention, Apparatus for treating a blemish having a furrow in a region of skin comprising: a bracing body having a surface that is placed contiguous with or in proximity to the region of skin, said bracing body having at least one air passage through which air between the skin region and the surface can be aspirated; and a vacuum pump coupled to the bracing body for aspirating air through the air passage so as to flatten the blemish to the bracing body surface.

Optionally, the bracing body comprises: a thin dielectric layer bonded to a conductor, wherein the bracing body surface is a surface of the dielectric layer not bonded to the conductor; and a power supply that applies a voltage difference between the conductor and the skin.

Optionally, the apparatus comprises a deferrable foil having an adhesive layer for bonding the foil to the region of skin and wherein when air is aspirated through the bracing body, the foil is flattened to the bracing body surface.

In some embodiments of the present invention the apparatus comprises: a foil having a dielectric layer bonded to a conducting layer, which foil is deformable so that the dielectric layer contacts skin in the furrow; and a power supply that generates a potential difference between the conducting layer and the skin so that electrostatic forces adhere the foil to the skin in the furrow and wherein when air is aspirated through the bracing body, the foil is flattened to the bracing body surface.

Alternatively or additionally the bracing body and the foil are optionally formed from a magnetized material and wherein once the foil is flattened to the bracing body surface, magnetic forces maintain the foil in contact with the bracing body surface.

In some embodiments of the present invention the bracing body is formed from a porous material and wherein the at least one air passage comprises air passages formed by pores in the material.

In some embodiments of the present invention the at least one air passage of the bracing body has particles on surfaces thereof that absorb a gas released through the skin.

There is further provided in accordance with an embodiment of the present invention an apparatus for treating a blemish having a furrow in a region of skin comprising: a foil comprising magnetic material deformable so as to contact skin in the furrow, which foil, when in contact with skin, adheres to the skin through the agency of an adhesive or electrostatic forces; a bracing body positioned in proximity to the foil; and a magnetic element that attracts the foil and flattens the foil to the bracing body surface. Optionally, the bracing comprises the magnetic element.

There is further provided in accordance with an embodiment of the present invention an apparatus for treating a skin blemish having a furrow comprising: a bracing body having a surface; an elastic foil having edges attached to the bracing body surface and a surface covered with an adhesive that faces away from the bracing body surface; and an air passage that communicates with a space between the foil and the bracing body surface, wherein when air is forced through the air passage, the foil distends away from the surface and adheres to skin in the furrow and when air is subsequently aspirated through the passage the foil substantially recovers its shape flattening to the bracing body surface and flattening thereby the blemish. Optionally, the apparatus comprises a pump that forces air through the air passage. Additionally or alternatively, the bracing body is a thin rectangular ribbon shaped body.

In some embodiments of the present invention the bracing body surface is formed so that the contour of the surface substantially matches the contour of the region of skin exclusive of contour features resulting from the blemishes in the skin.

In some embodiments of the present invention a blemish comprises a wrinkle.

There is further provided, in accordance with an embodiment of the present invention, a method for treating wrinkles in a region of skin comprising: heating the skin so that bent collagen fibers in the wrinkles become plastic; applying forces to the heated collagen fibers that tend to straighten the fibers; and cooling the collagen fibers while the forces are applied so that the collagen fibers retain a memory of their straightened configuration.

There is further provided, in accordance with an embodiment of the present invention, a bandage for protecting a region of skin comprising: a thin flexible layer of material formed with perforations for covering the skin region; and particles adhered to walls of the perforations formed from a material that absorbs a gas released by the body through the skin.

There is further provided, in accordance with an embodiment of the present invention, apparatus for administering a medication through a region of skin comprising: a conducting layer comprising at least one conductor; a thin dielectric layer bonded to the conducting layer; a power supply that applies a voltage between the conducting layer and the skin; a sensor that generates signals responsive to electromagnetic fields in the skin; a controller that receives the signals and uses the signals to determine a frequency of peristaltic waves in pores in the skin and modulates the voltage at the determined frequency, wherein the medication is sandwiched between the dielectric layer and the region of skin.

There is further provided, in accordance with an embodiment of the present invention a skin gripping apparatus for attracting and holding a region of skin comprising: a conducting layer comprising at least one conductor; a thin dielectric layer bonded to the conducting layer, which dielectric layer has a surface that is placed in contact with the region of skin so that the dielectric layer is between the conducting layer and the region of skin; and an electrical power supply that applies a potential difference between a conductor of the at least one conductor and the skin.

In some embodiments of the present invention, the power supply is mechanically integrated with the at least one conductor. Optionally, the power supply comprises a receptacle for holding a source of power. Alternatively, the power supply may comprise an antenna and circuitry for receiving energy radiated from a power source and processing received energy to provide a desired potential difference between the conducting layer and the skin. In some embodiments of the present invention the power supply is separate from and spatially removed from the at least one conductor.

In some embodiments of the present invention the dielectric layer is formed from a flexible dielectric material. In some embodiments of the present invention the dielectric layer is formed with local dislocations that breakdown electrically to permit currents to flow from the at least one electrode to the region of skin.

In some embodiments of the present invention the at least one conductor comprises at least one thin pliable conductor. In some embodiments of the present invention the conducting layer is bonded to an insulating substrate. Optionally, the insulating substrate is formed as a relatively thin flexible layer of insulating material.

Alternatively or additionally, the skin gripping apparatus is perforated with holes that extend from the region of skin to the air. Preferably, the walls of the holes are covered with an insulating material. Preferably, the insulating material covering the walls of the holes is bonded with the material in the insulating layer and the dielectric layer.

In some embodiments of the present invention a voltage applied to at least one conductor is a time varying voltage.

In some embodiments of the present invention the at least one conductor comprises a plurality of conductors. Optionally, the power supply applies a same voltage to each of the plurality of conductors. Alternatively, the power supply applies different voltages to at least two of the plurality of conductors.

In some embodiments of the present invention a skin gripping apparatus comprises a layer of piezoelectric material excitable to generate ultrasound waves that penetrate the region of skin. In some embodiments of the present invention a skin gripping apparatus comprises at least one heating element energizeable to heat the region of skin. Optioanlly, the heating element is a Peltier thermocouple that can operate as a cooling element.

There is further provided in accordance with an embodiment of the present invention a massaging apparatus for massaging the skin comprising: at least one skin gripping apparatus according to an embodiment of the present invention; and a means for producing motion of surface regions of the dielectric layer of the at least one skin gripping apparatus that contact the skin.

In some embodiments of the present invention, the means for providing motion comprises at least one handle for grasping the massaging apparatus and wherein motion of surface regions of the dielectric layer is produced by manually manipulating the at least one handle.

Alternatively or additionally, the dielectric surface of the at least one skin gripping apparatus is planar. Optionally, the layers of the skin gripping apparatus are stretchable and form a thin stretchable multilayer body. Optionally, the at least one handle comprises two handles and wherein the two handles are manually pulled away from each other to stretch the stretchable body and produce motion of the surface regions of the dielectric layer in contact with the skin.

There is further provided in accordance with an embodiment of the present invention a massaging apparatus for massaging the skin comprising: at least one skin gripping apparatus according to an embodiment of the present invention; and a controller that controls the power supply to apply voltage differences between the region of skin and different ones of the plurality of conductors to generate forces that produce motion of the surface regions of the dielectric.

In some embodiments of the present invention, a gripping apparatus comprises a layer of piezoelectric material excitable to generate ultrasound waves that penetrate the region of skin. Alternatively or additionally, a gripping apparatus comprises at least one heating element energizeable to heat the region of skin.

In some embodiments of the present invention, all the layers of the skin gripping apparatus are flexible and form a thin flexible multilayer body. Optionally, the at least one conductor is a plurality of parallel narrow rectangular strip conductors having short and long edges. In some embodiments of the present invention, the controller controls the power supply to apply voltage differences between the region of skin and different ones of the strip conductors to generate forces that produce wave motions in the flexible body that move back and forth in directions perpendicular to the long edges of the strip electrodes.

In some embodiments of the present invention dielectric surface of the at least one skin gripping apparatus is curved.

In some embodiments of the present invention the dielectric surface of the at least one skin gripping apparatus is cylindrical. In some embodiments of the present invention the directrix of the cylindrical surface is a circle. In some embodiments of the present invention the directrix of the cylindrical surface is an ellipse. In some embodiments of the present invention the directrix of the cylindrical surface is a polygon.

In some embodiments of the present invention the at least one skin gripping apparatus comprises a plurality of skin gripping apparatuses.

In some embodiments of the present invention the at least one skin gripping apparatus rolls along the region of the skin.

In some embodiments of the present invention the dielectric surface of at least one skin gripping apparatus comprises at least one protuberance. Optionally, the at least one protuberance comprises a plurality of dimple shaped protuberances. Alternatively or additionally, the at least one protuberance comprises a plurality of raised ribs that protrude from the surface.

There is further provided, in accordance with an embodiment of the present invention, a thermometer for measuring a person' temperature comprising: a skin gripping apparatus according to preferred embodiment of the present invention; a heat sensing element that is pressed by the skin gripping apparatus to a region of the person's skin that senses the person's temperature; and a display connected to the heat sensing element that displays the sensed temperature. Preferably, all the layers of the skin gripping apparatus are flexible. Preferably, the display is a flat panel display bonded to a layer of the skin gripping apparatus. Preferably, the flat panel display is a flexible flat panel display.

There is further provided, in accordance with an embodiment of the present invention, a nose guard for protecting a person's skin from the sun comprising: at least one skin gripping apparatus according to an embodiment of the present invention; a sun shade for the nose attached to the at least one skin gripping apparatus.

There is further provided, in accordance with an embodiment of the present invention, a decorative ornament to be worn by a person comprising: at least one skin gripping apparatus according to an embodiment of the present invention; and a decorative element attached to the at least one skin gripping apparatus. In some embodiments of the present invention, the dielectric layer of the at least one skin griping apparatus is cylindrical and the at least one conductor comprises a plurality of narrow rectangular conductors parallel to the axis of the cylindrical dielectric layer and the power supply electrifies conductors sequentially to cause the at least one skin gripping apparatus to roll along the person's skin. Alternatively or additionally, the decorative element may be a piece of jewelry.

There is further provided, in accordance with an embodiment of the present invention, a method of attaching an object to a person's skin comprising: attaching at least one conducting element coated with a thin layer of dielectric material to the object; placing the surface of the dielectric layer in contact with a region of skin; and applying a voltage between the electrode and the skin.

There is further provided, in accordance with an embodiment of the present invention, a method of massaging a person's skin comprising: placing a conductor having a surface coated with a thin layer of dielectric material on the person's skin so that the surface of the dielectric layer is contiguous with the skin; applying a voltage between the conductor and the skin; and moving the conductor.

There is further provided, in accordance with an embodiment of the present invention, a medication, which is in the form of a paste or a gel, to a person, the method comprising: sandwiching the medication between a surface of a conductor coated with a thin dielectric layer; and applying a voltage between the conductor and the skin. In some embodiments of the present invention, the method comprises controlling the voltage to control the rate at which the substance is perfused.

There is further provided, in accordance with an embodiment of the present invention, a method for treating blemishes on a region of person's skin comprising: placing on the region of skin an elastically stretchable, multi-layered body comprising a conducting layer coated with a thin dielectric layer so that the surface of the dielectric layer is contiguous with the skin; and repeatedly stretching and relaxing the layered body while the surface of the dielectric layer is in contact with the region of skin.

There is further provided, in accordance with an embodiment of the present invention, a method for treating blemishes on a region of a person's skin comprising: positioning a surface of a body so that the surface contacts or is proximal with the region skin; and attracting the region of skin to the surface of the body so that blemishes in the region of skin are flattened to the surface of the body.

In some embodiments of the present invention, positioning the surface comprises positioning a surface of a magnetized material proximal to the region of skin. Optionally, attracting the region of skin comprises adhering to the skin a magnetized foil that is attracted by magnetic forces to the surface of the magnetized material. Optionally, adhering to the skin comprises kneading the foil so that it contacts and sticks to skin in furrows of blemishes in the region of skin. Positioning a surface optionally comprises pressing a surface of a dielectric layer of a skin gripping apparatus according to an embodiment of the present invention so that it contacts the region of skin.

In some embodiments of the present invention, attracting the region of skin comprises: pressing the dielectric surface to the region of skin so that furrows of blemishes in the skin are flattened to the dielectric surface and contact the dielectric surface; and applying voltage to at least one electrode comprised in the gripping surface so that skin in the furrows is attracted to and sticks to the dielectric surface.

In some embodiments of the present invention, attracting the region of skin comprises aspirating air from between the region of skin and the surface.

There is further provided, in accordance with an embodiment of the present invention, a skin treatment device comprising: a skin gripping apparatus according to preferred embodiment of the present invention, having at least one conduit passing through layers in the skin gripping apparatus and terminating in an orifice in the dielectric layer; and a vacuum pump connected to the at least one conduit that aspirates air through the conduit. In some embodiments of the present invention, the pump is a manually operated pump. In some embodiments of the present invention, the pump is an electrically powered pump.

### BRIEF DESCRIPTION OF FIGURES

The invention will be more clearly understood by reference to the following description of embodiments thereof, read in conjunction with the figures attached hereto. In the figures, identical structures, elements or parts which appear in more than one figure are labeled with the same numeral in all the figures in which they appear. The figures are listed below and:

FIG. 1 schematically shows a skin-gripper attached to a region of a person's skin, in accordance with an embodiment of the present invention;

FIG. 2 schematically shows an electro-patch bandage comprising a skin-gripper having a flexible gripping body covering a wound, in accordance with an embodiment of the present invention;

FIGS. 3A and 3B schematically show different conductor configurations for the skin-gripper shown in FIG. 2, in accordance with an embodiment of the present invention;

FIG. 4A schematically illustrates perfusing a medication through a person's skin using a perfusion skin-gripper in accordance with an embodiment of the present invention;

FIG. 4B schematically shows a perfusion skin-gripper having a sensor for sensing electromagnetic fields in skin to which the perfusion sensor is applied, in accordance with an embodiment of the present invention;

FIG. 5 schematically shows a massager comprising a skin-gripper having a curved gripping surface being used to massage a person's skin, in accordance with an embodiment of the present invention;

FIG. 6 schematically shows a massager comprising a circularly cylindrical gripping surface that is rolled over a person's skin to massage the skin, in accordance with an embodiment of the present invention;

FIGS. 7A and 7B schematically show in different perspective views a stretch massager comprising a stretchable skin-gripper, in accordance with an embodiment of the present invention;

FIGS. 7C-7E show the stretch massager shown in FIGS. 7A and 7B being used to treat wrinkles, in accordance with an embodiment of the present invention;

FIGS. 8A and 8B schematically show the use of a thin magnetized wrinkle foil for treating wrinkles, in accordance with an embodiment of the present invention;

FIG. 8C schematically shows a wrinkle foil being used with a bracing layer in which vacuum is used to flatten the wrinkle foil to the bracing layer, in accordance with an embodiment of the present invention;

FIGS. 8D and 8E schematically show a wrinkle foil that is adhered to a region of skin using electrostatic forces, in accordance with an embodiment of the present invention;

FIG. 8F schematically shows a woman using a wrinkle foil, in accordance with an embodiment of the present invention;

FIGS. 9A-9C schematically show another wrinkle foil used to treat a wrinkle, in accordance with an embodiment of the present invention;

FIG. 9D schematically shows the wrinkle foil shown in FIGS. 9A-9C being used with a perfusion skin-gripper, in accordance with an embodiment of the present invention;

Fig. 10A shows a massager pattern massager comprising a flexible skin-gripper, in accordance with an embodiment of the present invention;

FIGS. 10B-l0D schematically illustrate using the skin-gripper shown in Fig. 1OA to massage a region of a person's skin, in accordance with an embodiment of the present invention;

FIG. 11 schematically shows a motile massager moving over a person's skin, in accordance with an embodiment of the present invention;

FIGS. 12A and 12B schematically show two other motile massagers, in accordance with an embodiment of the present invention;

FIG. 13A and 13B schematically show another motile massager being used to massage a person's skin, in accordance with an embodiment of the present invention;

FIG. 14 schematically shows a massager comprising ultrasound transducers for treating a region of skin with ultrasound radiation, in accordance with an embodiment of the present invention;

FIG. 15A schematically shows a vacuum massager being used to treat a person's skin, in accordance with an embodiment of the present invention;

FIGS. 15B and 15C schematically show features of the construction of the vacuum massager shown in FIG. 15A, in accordance with an embodiment of the present invention;

FIG. 16 schematically shows a thermometer, being held to a person's forehead to measure the person's temperature, in accordance with an embodiment of the present invention;

FIG. 17 schematically shows a nose guard comprising a skin-gripper for protecting a person's nose against sunburn, in accordance with an embodiment of the present invention;

FIG. 18 schematically shows a piece of jewelry held in place on a person's neck by a skin-gripper in accordance with an embodiment of the present invention; and

FIG. 19 shows schematically a “moving skin decoration” that comprises a motile skin-gripper, in accordance with an embodiment of the present invention.

### DETAILED DESCRIPTION OF EXAMPLES OF EMBODIMENTS

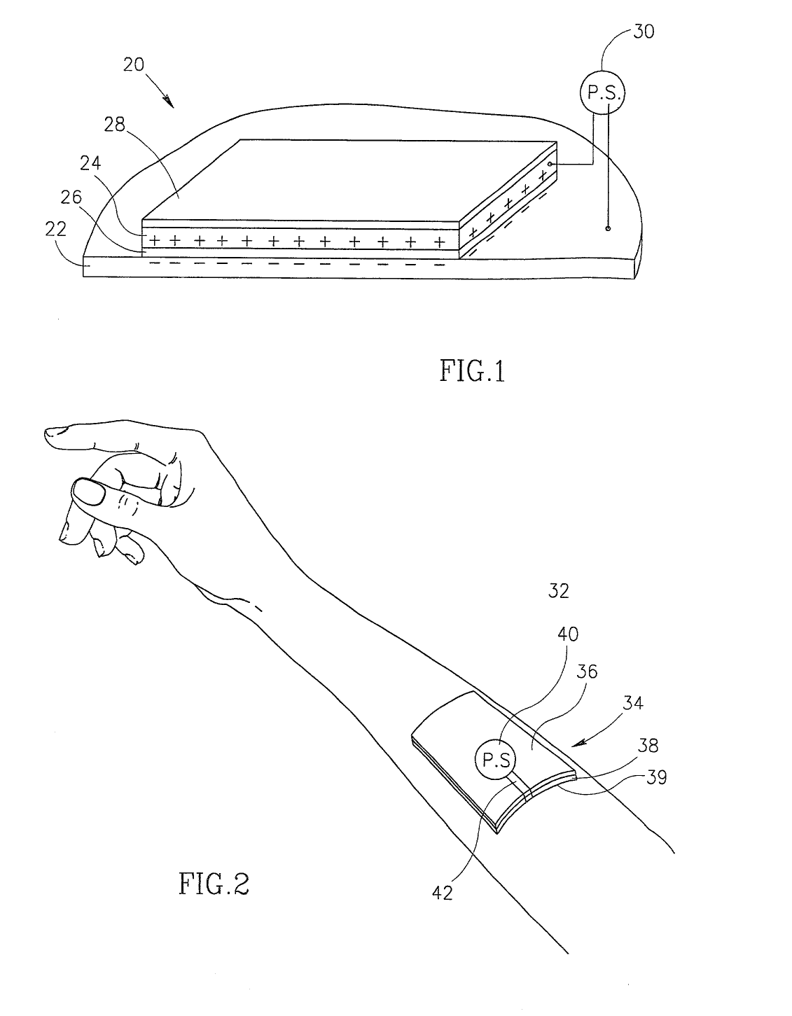

FIG. 1 schematically shows a skin-gripper 20, in accordance with an embodiment of the present invention, in contact with a person's skin 22. Skin-gripper 20 and skin 22 are shown in a cutaway perspective view. The dimensions of elements and features shown in FIG. 1 and figures that follow are not necessarily to scale and are chosen for convenience of presentation.

Skin-gripper 20 comprises a conducting layer 24 bonded to a layer 26 of dielectric material. Optionally, the surface of conducting layer 24 not bonded with dielectric layer 26 is protected with a layer 28 of insulating material. A surface of dielectric layer 26 is in contact with skin 22. The surface is a gripping surface of skin-gripper 20. A power supply 30 generates a voltage difference V, i.e. a gripping voltage, between conducting layer 24 and skin 22.

Whereas power supply 30 is shown separate from the bonded layers of skin-gripper 20, in some embodiments of the present invention, power supply 30 is attached to or integral with the layers. In some embodiments of the present invention energy is supplied to power supply 30 by a battery. In some embodiments of the present invention energy is supplied to power supply 30 from a photovoltaic converter or other source.

Skin 22, conducting layer 24 and dielectric insulating layer 26 function as a parallel plate capacitor charged by power supply 30 to the voltage V. As a result of the voltage difference between conducting layer 24 and skin 22, substantially equal and opposite surface charges are generated on conducting layer 24 and a region of skin 22 opposite conducting layer 24. In FIG. 1 the charges are represented by plus and minus signs and it is assumed, by way of example, that conducting layer 24 is charged positively.

The magnitude of the surface charge densities and the force of attraction between conducting layer 24 and skin 22 resulting from the charge densities may be calculated from the usual capacitor formulae. The magnitude of the generated surface charge densities is equal to κε₀ V/d, where d and κ are the thickness and dielectric constant respectively of dielectric layer 26 and ε₀ is the permittivity of free space. The force of attraction per cm² of surface area of conducting layer 24 is (ε_{0/}2)(κV/d)². By way of example, assuming V=100 volts, κ=20, and d=10 microns, then the force between conducting layer 24 and skin 22 is 20 N/cm² of surface area of conducting layer 24. In other words, a pull force of about 2 kg is needed to separate skin-gripper 20 from skin 22 for every cm² of contact between dielectric layer 26 and skin 22.

It should be realized that once conducting layer 24 is charged and positioned on skin 22, conducting layer 24 discharges very slowly. Very little power therefore has to be supplied to maintain a given potential difference between conducting layer 24 and skin 22. Since power requirements of skin-gripper 20 are low, it is practical to supply power needed to maintain a given potential difference between conducting layer 24 and skin 22 by radiating power to skin-gripper 20. Therefore, in some embodiments of the present invention power supply 40 is not wired to a source of energy such as a battery or a wall outlet. Instead energy is radiated to power supply 30 from an appropriate energy source. In these embodiments of the present invention, skin-gripper 20 comprises antenna and circuitry for receiving the radiated energy and processing it to maintain a given potential between conducting layer 24 and skin 22. It is to be noted that antennae and circuitry appropriate for receiving and processing energy for use with skin-gripper 20 are well known in the art.

It should also be realized, that because of the low power requirements of skin-gripper 20, in some case it is possible to place skin-gripper 20 on skin 22, electrify it to a gripping voltage and then disconnect power supply 30. Skin-gripper 20 will remain attached to the skin with a strong gripping force for a substantial period of time since charge on conducting layer 24 leaks off very slowly.

FIG. 2 shows an electro-patch bandage 32 covering a wound (not shown) on a person's arm. Electro-patch bandage 32 optionally comprises a pliable skin-gripper 34. In some embodiments of the present invention skin-gripper 34 is formed from a pliable conducting layer (not shown) comprising at least one conductor (not shown) sandwiched between a pliable insulating layer 36 and a thin dielectric layer 38. (In the perspective of FIG. 2 the conducting layer is not seen because layers 34 and 36, by way of example, extend beyond the edges of the conducting layer) A surface 39 (only an edge of which is shown) of dielectric layer 38 contacts the person's skin. Surface 39 is a gripping surface of skin-gripper 34.

In some embodiments of the present invention, a power supply 40 is integrated with skin-gripper 34 and is, optionally, mounted on insulating layer 36. Power supply 40 generates a gripping voltage between the conducting layer and the skin on the person's arm. Optionally, power supply 40 comprises an appropriate switch, such as a pressure-activated switch, which is used to turn on power supply 40 when skin-gripper 34 is placed on the arm. A conducting ground strip 42 extends from power supply 40 so that a portion of ground strip 42 lies over gripping surface 39 of dielectric layer 38. Ground strip 42 assures proper electrical connection between power supply 40 and the skin when electro-patch bandage 32 is placed on the skin. Optionally, ground strip 42 is silver-plated or made from silver, which is known to have beneficial anti-bacterial activity.

Because of its pliability, electro-patch bandage 32 conforms to the shape of a part of the body to which it is applied and thereby provides protection of a wound that it covers from ingress of contaminants.

The dimensions and shape of electro-patch bandage 32 have been chosen for ease and clarity of presentation and are not intended to imply a limitation of the invention. Different sizes and shapes of electro-patch bandages, in accordance with embodiments of the present invention, are possible and can be advantageous. The size and shape of an electro-patch bandage, in accordance with an embodiment of the present invention, can be tailored to the size and extent of a wound it is intended to cover.

In some embodiments of the present invention, the conducting layer in an electro-patch bandage comprises an array having a plurality of conductors. In some embodiments of the present invention conductors in the array are electrified by a power supply so as to generate an electrostatic field that has substantial components parallel to a region of skin to which the electro-patch bandage is applied. In some embodiments of the present invention, adjacent conductors in the array are charged by the power supply to opposite polarity voltages to generate the electric field. In some embodiments of the present invention, a varying voltage is applied to at least one conductor in the array so as to produce a time dependent electric field, such as an harmonically varying electric field, in the region of a wound covered by the electro-patch bandage. Time dependent electric fields cause motion of electrolytes in the skin and in tissue below the skin that generates internal heating of tissue in the region of the wound. In addition the fields create time varying pressure gradients that massage tissue in the region of the wound and increase blood flow to the injured region. Both the heating and massaging promote healing.

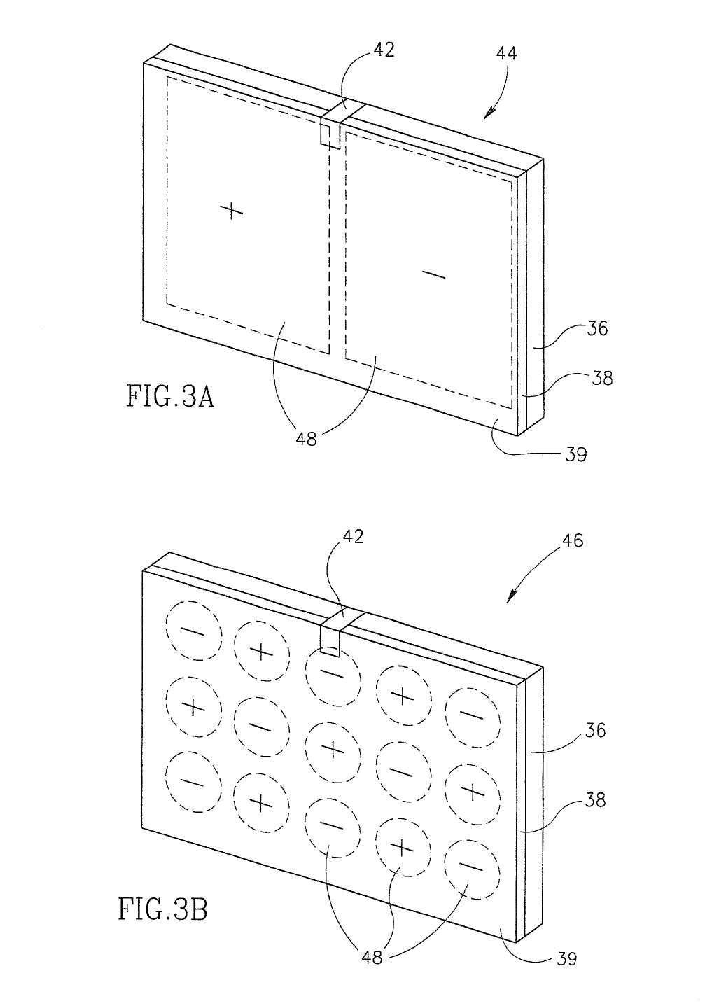

FIGS. 3A and 3B schematically show two electro-patch bandages 44 and 46 that are variations of electro-patch bandage 32 shown in FIG. 2. In FIGS. 3A and 3B gripping surfaces 39 of electro-patch bandage 44 and 46 and the portion of their ground strips 42 that lie on the gripping surfaces are shown. Electro patch bandages 44 and 46 exhibit two different arrays of conductors 48, in accordance with embodiments of the present invention. Conductors 48, are shown in ghost lines because they are covered by their respective dielectric layers 38 and are not normally visible.

Conductors 48 are, optionally, individually electrifiable by power supply 40 (FIG. 2), which is grounded to the skin through ground strip 42, to generate different spatially and temporally varying electric fields. Conductors 48 in FIGS. 3A and FIG. 3B are shown with plus and minus signs indicating a pattern of electrification of conductors 48 that is used to generate electrostatic fields having components parallel to the skin. Other arrays of conductors and electrification patterns can be advantageous and will occur to persons of the art.

It is to be noted that static and time varying electric fields and electric fields with components parallel to the skin in a region of a skin wound are known to be beneficial to the healing process of wounds. U.S. Pat. No. 4,142,521 to Konikoff, the disclosure of which is incorporated herein by reference, describes benefits to healing that result from the application of electrostatic fields to wounds. The patent describes a bandage comprising an electret for generating an electric field in the vicinity of a wound. The electret and bandage are held in place with gummed tape. U.S. Pat. No. 4,911,688 to J. P. Jones, the disclosure of which is incorporated herein by reference, describes covering a wound with a bandage that maintains a liquid layer over the wound. The bandage, which is held in place with an adhesive, comprises a means for producing an electric field in the liquid to enhance healing of the wound. Professor Joseph. W. Vanable Jr. at internet site “www.bio.purue.edu/Bioweb/people/faculty/Vanable”, reports on research indicating the importance of electric fields on wound healing in update of Jul. 10, 1998 of the site, the disclosure of which is incorporated herein by reference.

In some embodiments of the present invention the dielectric layer in an electro-patch bandage, such as dielectric layer 38 shown in FIGS. 2, 3A and 3B, is formed with a substantially uniform distribution of local dislocations in its structure. The dislocations are formed using methods known in the art such as by irradiating the dielectric layer with electrons or alpha particles. At appropriate gripping voltages, the dielectric layer breaks down in the regions of the local dislocations to enable small electrical micro-current pulses having current magnitudes ranging from a few pico-amperes to nano-amperes to flow between the conducting layer of the bandage and skin to which the electro-patch bandage is applied.

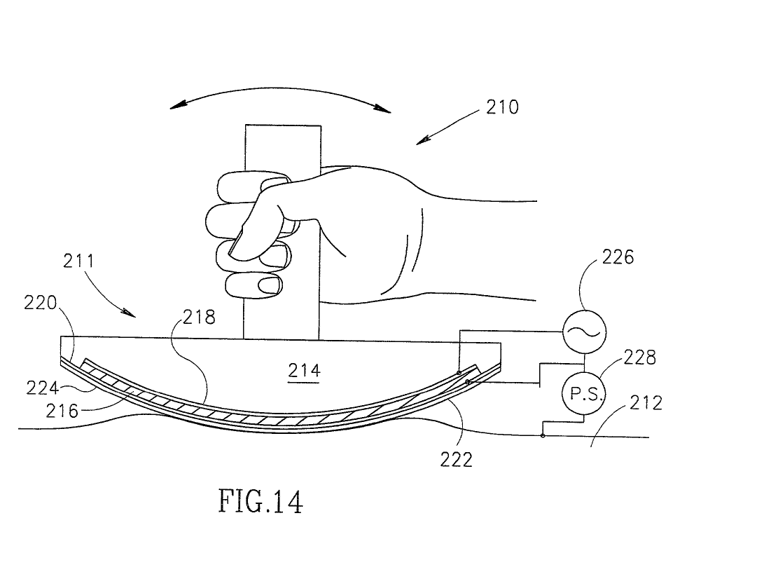

Some electro-patch bandages in accordance with embodiments of the present invention comprise a layer of piezoelectric material and a power supply for exciting ultrasound vibrations in the piezoelectric layer. The vibrations generate ultrasound waves that radiate into tissue in the region of a wound protected by the bandage. The piezoelectric layer and power supply are incorporated in the electro-patch bandage in a manner similar to the way in which incorporation of a piezoelectric layer in a massager, in accordance with an embodiment of the present invention, is illustrated in FIG. 14 discussed below.

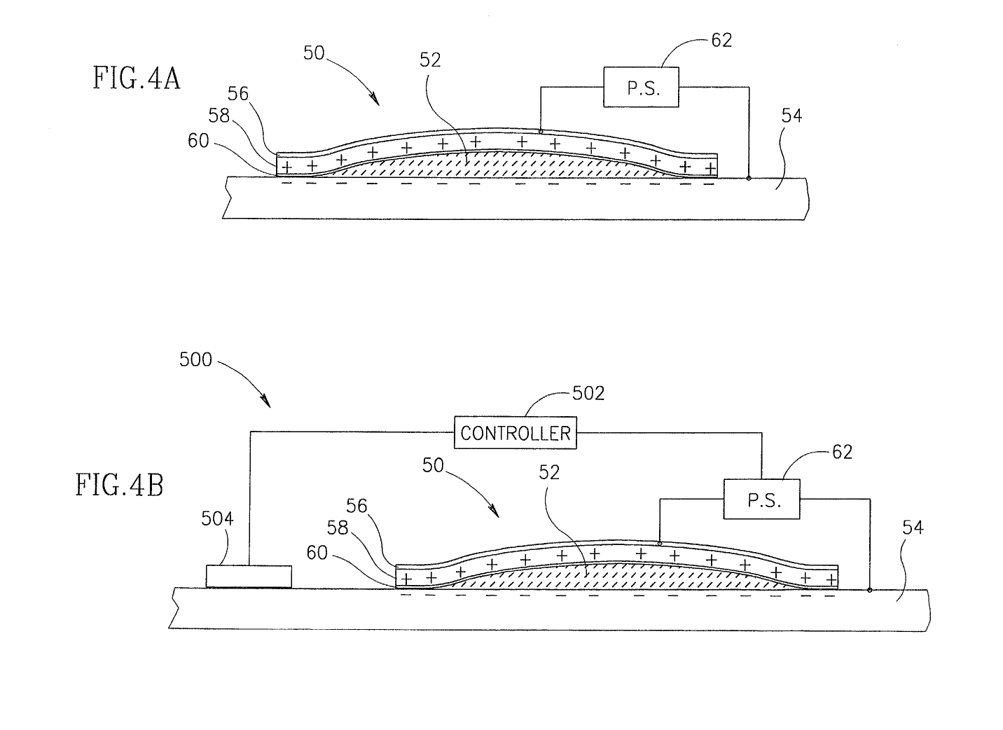

FIG. 4A schematically shows a “perfusion” skin-gripper 50 in a cross-section view, used to perfuse a medicine 52 into the body of a person through the person's skin 54. In some embodiments of the present invention, perfusion skin-gripper 50 has a structure similar to that of electro-patch bandage 32 shown in FIG. 2 and comprises a flexible insulating layer 56, a flexible conducting layer 58 optionally comprising a single conductor and a thin dielectric layer 60. A power supply 62 applies a potential difference between conducting layer 58 and skin 54. The potential difference generates surface charges on conductor 58 and skin 54, shown respectively with plus and minus signs, that result in a relatively large electric field and large attractive forces between perfusion skin-gripper 50 and skin 54.

Medication 52 is preferably applied in a thin layer (shown greatly exaggerated in thickness in FIG. 4A) to dielectric layer 60. Optionally, medication 52 is a dielectric so that the amount by which the presence of medication 52 reduces the electric field at the surface of the skin is mitigated. The forces of attraction between perfusion skin-gripper 50 and skin 54 perfuse medication through skin 54. Preferably, medication 52 is restricted to a central portion of the area of the surface of dielectric layer 60. This tends to prevent medication 52 from being squeezed out along edges of perfusion skin-gripper 50. If medication 52 comprises charged or polar molecules or particles, perfusion is enhanced by the electric field between perfusion skin-gripper 50 and skin 54. Perfuision of a medication using an electrophoretic effect is described in U.S. Pat. No. 4,767,401 to M. Seiderman, the disclosure of which is incorporated herein by reference. The patent describes “iontophoretic administration of ionizable or polar medicaments to a mammalian body” using a bandage that is held in place on the skin with an adhesive. The bandage comprises a metal electrode that in contact with the skin generates a voltaic electric field that perfuses the medication.

In some embodiments of the present invention, a perfusion skin-gripper comprises a sensor that senses electrical fields in the skin and/or underlying tissue and a controller. The sensor transmits signals responsive to electrical fields in a region of skin to which the perfusion skin-gripper is applied and/or electrical fields in tissue underlying the region of skin to the controller. The controller processes the signals and controls voltage between the perfusion skin-gripper and the skin responsive to the processed signals.

FIG. 4B schematically shows a perfusion skin-gripper 500 being used to perfuse a medication 52 through a region of skin 54. Perfusion skin-gripper 500 comprises perfusion skin-gripper 50 shown in FIG. 4A, a controller 502 and a sensor 504 for sensing electrical fields in the skin and/or underlying. Numerous different types of sensors and methods for detecting electromagnetic fields in skin and muscles known in the art are suitable for use in the practice of the present invention. Sensor 504 generates signals responsive to electromagnetic fields in skin 54 and/or underlying tissue and transmits the signals to controller 502. Controller 502 processes the signals and controls power supply 62 to apply a voltage difference between electrode 56 and skin 54 that is modulated responsive to the processed signals.

In some embodiments of the present invention, controller 502 processes signals from sensor 504 to determine a frequency of peristalsis of pores (not shown) in skin 54 and controls power supply 62 to apply a voltage between electrode 56 and skin 54 that is modulated at the peristaltic frequency. For example, controller 502 might control power supply 62 to apply a voltage difference between electrode 56 and skin 54 that comprises a DC voltage perturbed by an AC voltage having a frequency equal to the peristaltic frequency. Electrical stimulation of skin 54 at a “peristaltic frequency” tends to increase the rate of transdermal perfusion of medication 52. In some embodiments of the present invention, a perfusion skin-gripper comprises a piezoelectric vibrator (not shown) and controller 502 controls the vibrator to stimulate the region of skin with ultrasound waves modulated at the peristaltic frequency.

Preferably, during measurements of peristaltic frequencies, the controller grounds electrode 58 to skin 54 to diminish electromagnetic background noise that might interfere with the frequency measurements. Grounding of the electrode is performed for periods of time sufficiently short so that the perfusion skin-gripper maintains contact with the skin.

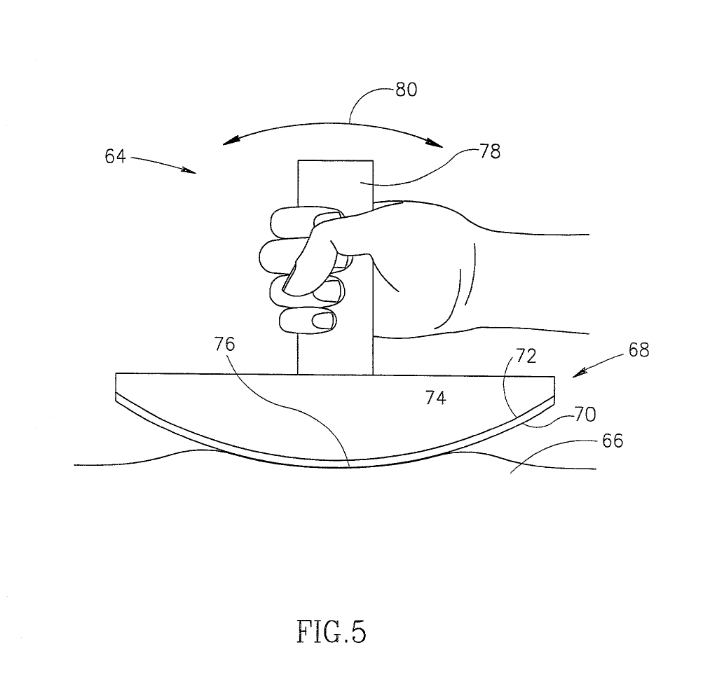

FIG. 5 schematically shows a cross section view a massager 64, in accordance with an embodiment of the present invention, being used to massage a person's skin 66. Massager 64 optionally comprises a skin-gripper 68 having a curved gripping surface 70. Gripping surface 70 may be formed by forming or adhering a conducting layer 72 on a curved substrate body 74 and laminating conducting layer 72 with a thin layer 76 (shown greatly exaggerated in thickness) of dielectric material. In some embodiments of the present invention substrate body 74 is mounted to a handle 78 in which, optionally, a power supply (not shown) for electrifying conducting layer 72 with a gripping voltage, is mounted. In some embodiments of the present invention, a conductor (not shown) is located on a curved edge of gripping surface 70 for providing electrical contact between the power supply and skin 66.

To operate massager 64, a gripping voltage is applied between conducting layer 72 and skin 66 such that gripping surface 70 is held firmly to skin 66. The massager is then and rocked back and forth as indicated by double-headed arrow 80. Because of the curved shape of gripping surface 70, as gripping surface 70 is rocked, it exercises skin 66 by repeatedly gripping, stretching and then releasing different regions of the skin to which it is pressed. In addition, the rocking motion and pressure with which gripping surface 70 is pressed to skin 66 mechanically massages skin 66 and tissue below the skin.

As in the case with electro-patch bandages, in some embodiments of the present invention dielectric layer 76 is formed with a substantially uniform distribution of local dislocations in its structure. The dislocations promote the flow of small electrical micro-current pulses between conducting layer 72 and skin 66.

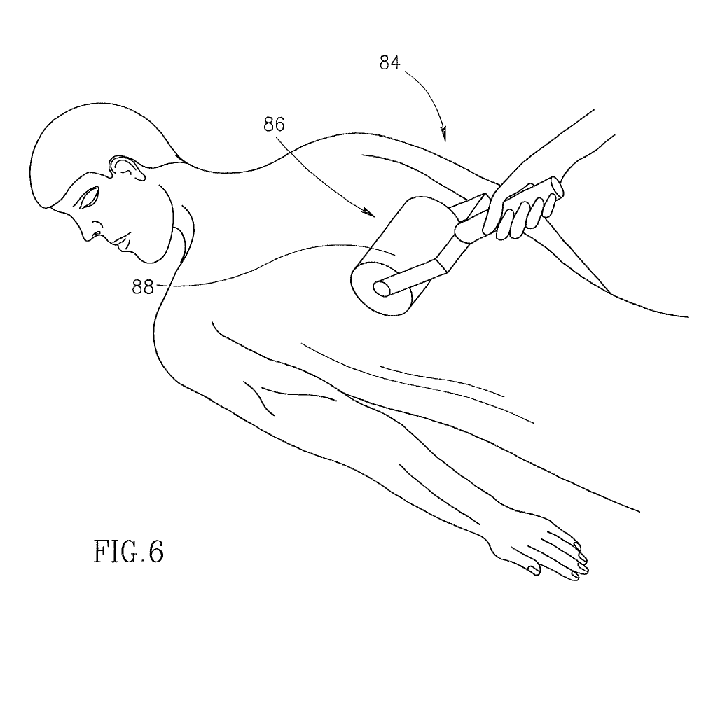

FIG. 6 shows another massager 84 in accordance with an embodiment of the present invention being used to massage a region of skin on a person's back. Massager 84 comprises a skin-gripper 86 having a circularly cylindrical gripping surface 88 which is rolled back and forth over the region of skin being massaged. Attraction between gripping surface 88 and the skin exercises the skin in much the same way that massager 64 exercises the skin.

Massagers, in accordance with embodiments of the present invention that operate similarly to massagers 64 and 84, but which comprise skin-grippers having gripping surfaces different from those of massagers 64 and 84 are possible and can be advantageous. For example, gripping surface 70 of massager 64 may be planar (in which case the gripping surface does not actually “rock” back and forth but tilts back and forth while holding and pulling the skin) or have a corrugated shape. Or it may be formed with a pattern of protruding dimples or parallel ribs that protrude from gripping surface 70. Circularly cylindrical gripping surface 88 of massager 84 may be a cylindrical surface defined by a directrix that is an ellipse, a polygon or an irregular closed curve. In some embodiments of the present invention, the dimensions and shape of gripping surface 88 may vary along its axis. Other massagers, in accordance with embodiments of the present invention comprise a plurality of separate cylindrical gripping surfaces, i.e. “wheels”, that are mounted on a common axis and rolled over the skin in similar fashion to the way in which massager 84 is rolled over the skin. Still other geometries for massagers will occur to persons of the art. It should be noted that whereas many different shapes and configurations of shapes are possible for a gripping surface of a massager, in accordance with embodiments of the present invention, different shaped gripping surfaces may grip skin with different forces for a same applied voltage between the skin and the conducing layer in the massager.

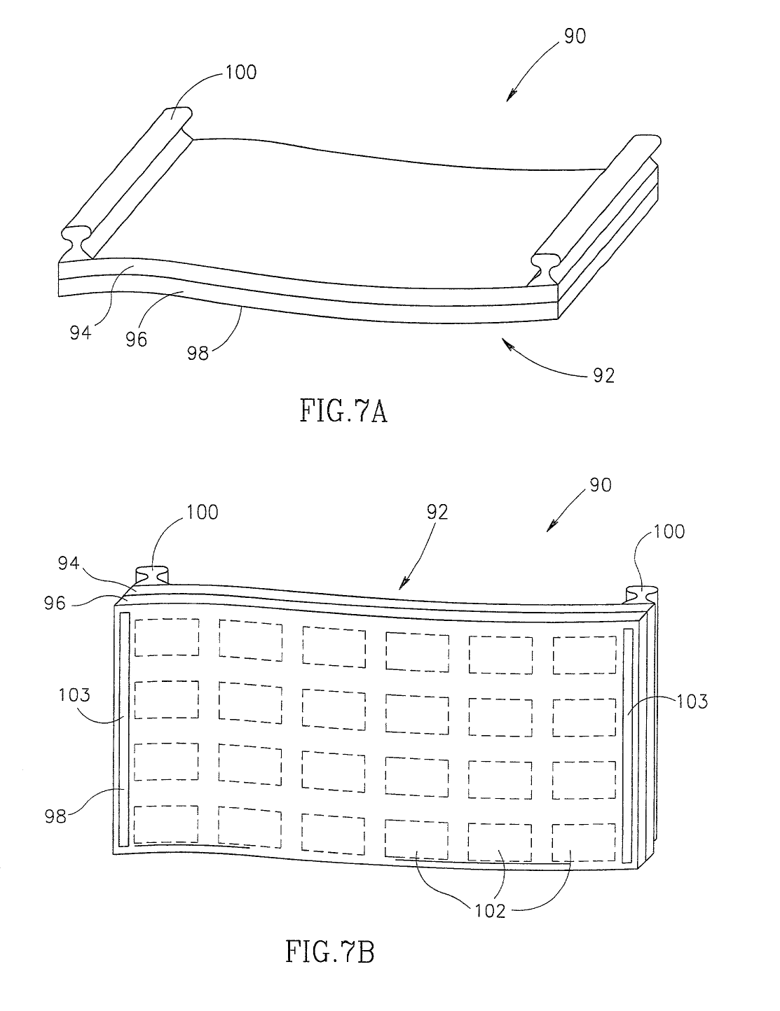

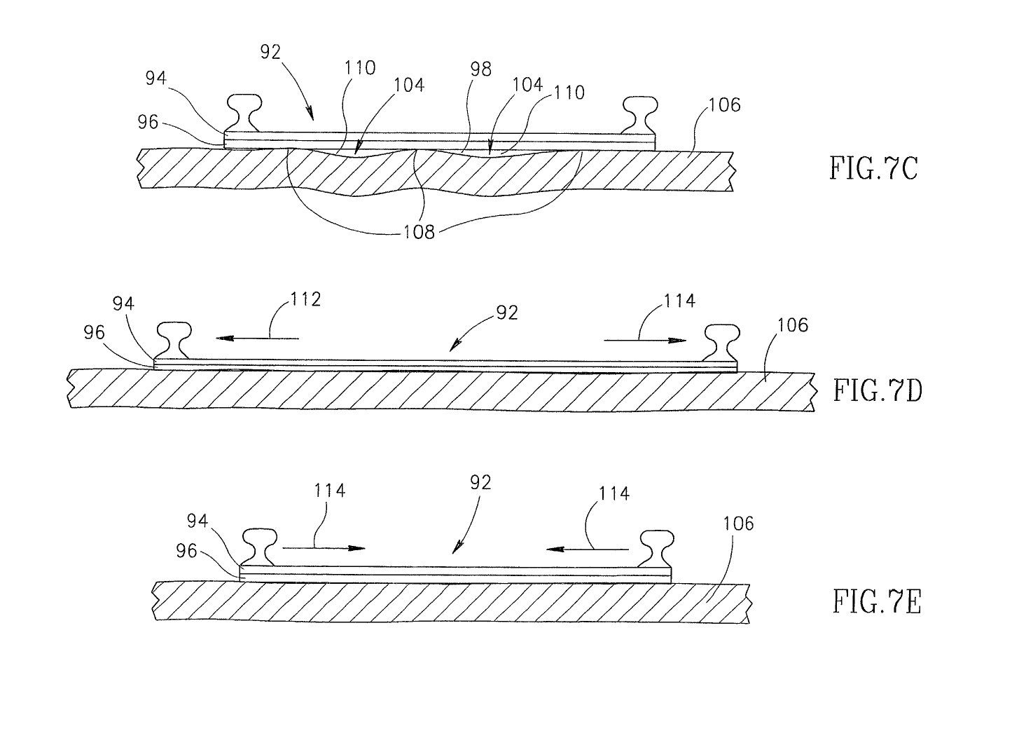

FIGS. 7A and 7B schematically show a massager 90, hereinafter referred to as “stretch massager 90”, comprising a relatively thin stretchable skin-gripper 92 and two handles 100.

FIG. 7A shows stretch massager 90 in a side perspective view. In accordance with an embodiment of the present invention, skin-gripper 92 comprises a stretchable, insulating substrate layer 94 and a conducting layer (not shown) optionally comprising a plurality of conductors bonded to substrate layer 94 in a dense regular array. A thin stretchable dielectric layer 96 is bonded to the conducting layer. A surface 98 of dielectric layer 96 is a gripping surface of skin-gripper 92. Handles 100 are, optionally, attached to skin-gripper 92 so as to enable skin-gripper 92 to be easily stretched manually.

FIG. 7B shows stretch massager 90 in a perspective view as seen from the gripping surface 98 side of skin-gripper 92. In FIG. 7B conductors 102 in the conducting layer of skin-gripper 92 are shown. Conductors 102 are shown in ghost lines because they are located under dielectric layer 96 and are not normally seen in the perspective of FIG. 7B. A power supply (not shown) for applying a gripping voltage to conductors 102 is appropriately wired to each conductor 102, using methods known in the art so that when skin-gripper 92 is stretched, electrical contact between the power supply and each conductor 102 is maintained. Optionally, the power supply is grounded to ground conductors 103 that are located on the surface of dielectric layer 96 under each handle 100. When stretch massager 90 is used to treat a person's skin, ground electrodes 100 are pressed to the skin, thereby grounding the power supply to the skin. In some embodiments of the present invention, the power supply is grounded to the skin through a conductor attached to the skin using methods known in the art or by using a skin-gripper in accordance with an embodiment of the present invention that presses the conductor to the skin.

Whereas conductors 102 in FIG. 7B are square, conductors in a stretchable skin-gripper, in accordance with an embodiment of the present invention, may have various shapes and be arrayed in different patterns. For example, in some embodiments of the present invention, conductors in a stretchable skin-gripper are small thin circular, hexagonal or diamond shaped conductors mounted to a substrate in a densely packed regular array. In accordance with another embodiment of the present invention the conductors are parallel closely spaced narrow rectangular conductors. In yet other embodiments of the present invention the conductors are arranged in a closely woven conducting mesh that is itself stretchable. Still other geometries and arrays of conductors are possible and can be advantageous and these will occur to a person of the art.

FIGS. 7C-7E schematically illustrate stretch massager 90 being used to treat wrinkles 104 (shown in FIG. 7C) in a region of skin 106, in accordance with an embodiment of the present invention. In FIGS. 7C-7E skin-gripper 92 and skin 106 are shown in cross-section. Wrinkles 104 are defined by ridges 108 and furrows 110.

In FIG. 7C skin-gripper 92 is shown positioned on skin 106 in an “unstretched” state and with conductors 102 raised to a gripping voltage by the power supply. Skin on ridges 108 of wrinkles 104 contact gripping surface 98 and is held fast to gripping surface 98. On the other hand, skin in furrows 110 of wrinkles 104 is not in contact with gripping surface 98.

In FIG. 7D skin-gripper 92 is stretched in directions indicated by arrows 112. Ridges 108 of wrinkles 104, which are held fast to gripping surface 98, are pulled apart. As a result, furrows 110 of wrinkles 104 are flattened out and skin in the flattened furrows comes into contact with gripping surface 98. Once in contact with gripping surface 98, skin in flattened furrow 110 is held fast by gripping surface 98.

In FIG. 7E skin-gripper 92 has been allowed to return to its unstretched state as indicated by arrows 114. However, even after skin-gripper 92 has been returned to its unstretched state, skin in furrows 110 that has come in contact with gripping surface 98 remains in contact with gripping surface 98. As a result, even when skin-gripper 92 is released to its unstretched state, wrinkles 104 remain flattened to gripping surface 98.

By repeatedly stretching and releasing skin-gripper 92 skin 106 is exercised and blood flow to skin 106 is increased. The exercise and stimulation improves skin tone and is conducive to the reduction of wrinkling. In some embodiments of the present invention, as in the case with massagers described above, skin-gripper 92 is constructed so that skin 106 is also stimulated by micro-currents that flow through dielectric layer 96.

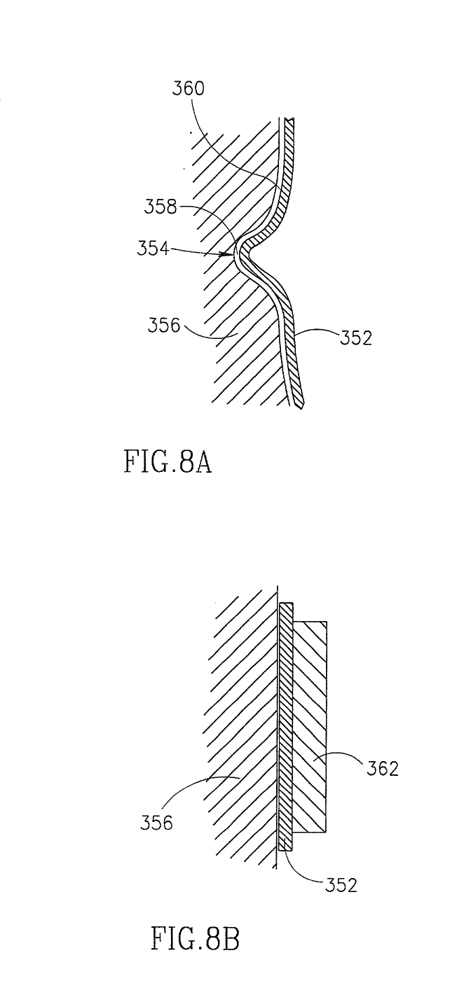

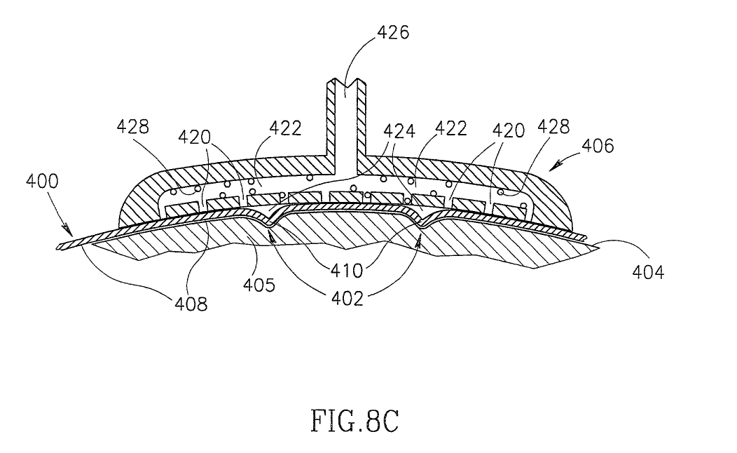

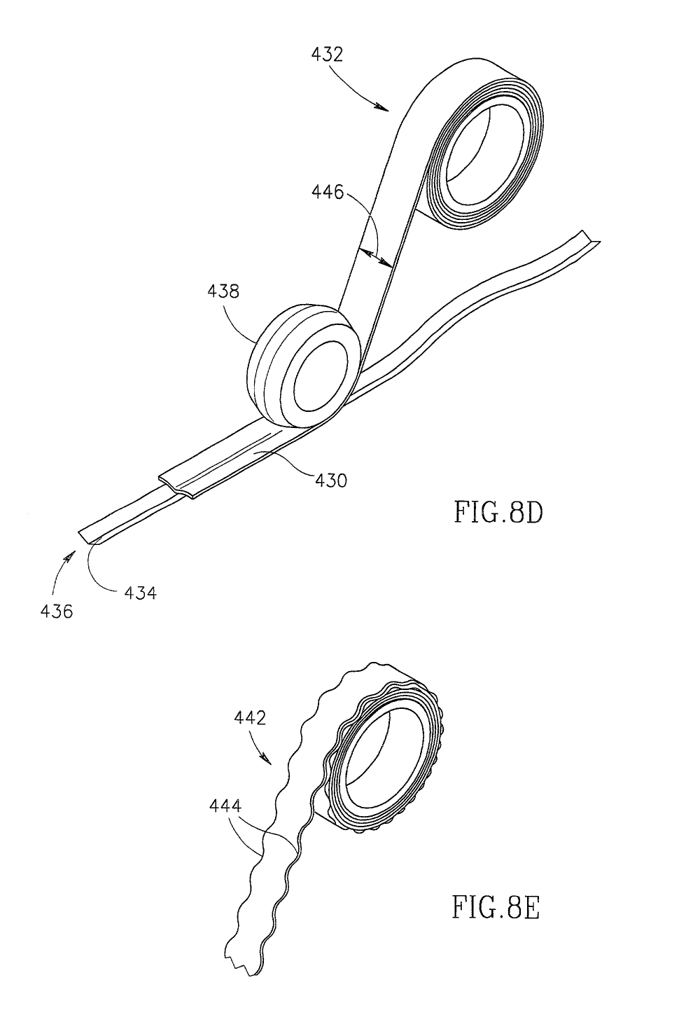

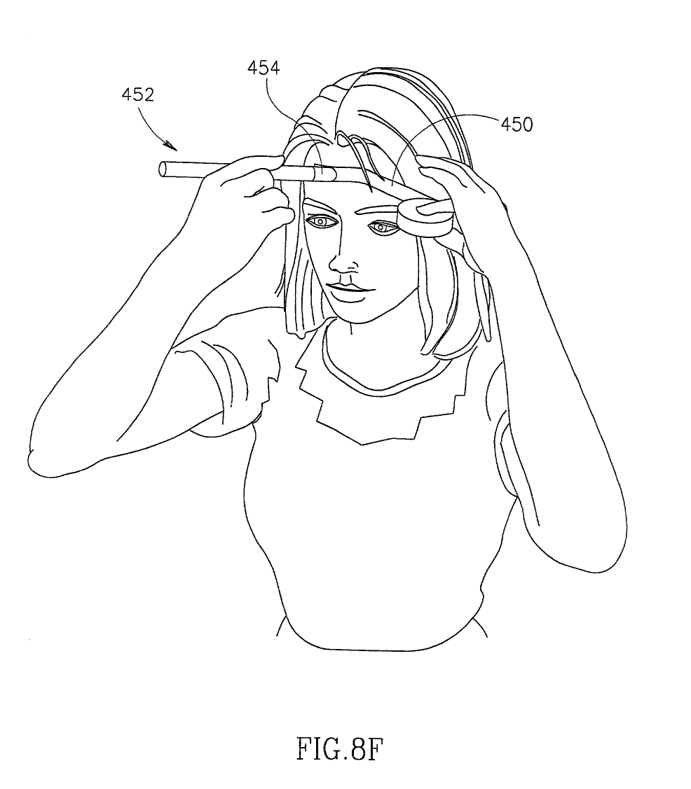

In some embodiments of the present invention magnetic fields are used to attract regions of skin located in furrows of wrinkles and smooth thereby the wrinkles by flattening them to an appropriate surface. FIGS. 8A and 8B schematically illustrate a method for treating wrinkles using a thin magnetic foil 352, i.e. a “wrinkle foil”, and a relatively thick magnetic bracing layer 362 to treat wrinkles 354, one of which is shown in FIGS. 8A and 8B, on a region of skin 356. Wrinkle 354 shown in 8A and 8B is defined by a furrow 358. In FIGS. 8A and 8B skin 356, foil 350 and bracing layer 352 are shown in cross section.

Referring to FIG. 8A, a surface 360 of magnetic wrinkle foil 352 covered with an adhesive material (not shown) is placed in contact with and kneaded to skin 354. As a result of the adhesive and kneading, surface 360 sticks strongly to skin 356. The thickness of wrinkle foil 352 and its Young's modulus are determined, in accordance with an embodiment of the present invention, so that when kneaded, wrinkle foil 352 molds to furrow 358 of wrinkle 354 in skin 356. FIG. 8A schematically shows wrinkle foil 352 after it has been kneaded and formed to furrow 358 of wrinkle 354. Surface 360 is shown distanced from skin 356 for convenience of presentation.

An appropriate foil suitable for use as wrinkle foil 352, in accordance with an embodiment of the present invention, is, by way of example, a 25 micron foil formed from a flexible plastic having a Young' modulus equal to about 10⁹ Pa (pascal=1 newton/m²), which is magnetized using methods known in the art. Such foils are known in the art and are manufactured, for example, by Bloomer Plastics Inc. of Bloomer, Wis., USA. Young's modulus for skin typically has values in a range from 10⁶ to 10⁷ Pa. The average thickness of skin is about 1 mm. A plastic foil having the noted characteristics therefore has substantially negligible bending rigidity compared to skin and can therefore relatively easily be kneaded into wrinkles in the skin.

After wrinkle foil 352 is intimately stuck to skin 356 as described above, bracing layer 362 is pressed to the foil. Magnetic forces between wrinkle foil 352 and bracing layer 362 attract wrinkle foil 352 to bracing layer 362 and flatten wrinkle foil 352, and thereby furrow 358 of wrinkle 354 to bracing layer 362. FIG. 8B shows bracing layer 362 pressed to wrinkle foil 352, and wrinkle foil 352 and wrinkle 354 flattened to bracing layer 362. Bracing layer 362 and wrinkle foil 352 are left in place for a convenient period of time, such as the duration of an afternoon nap or of a typical duration of a mudpack application received in a beauty parlor. In some embodiments of the present invention, wrinkle foil 352 and bracing layer 362 are perforated or formed from suitable porous materials so that the skin can 37 breath” while being treated. Bracing layer 362 and wrinkle foil 352 are periodically applied to skin 354 to “iron out” and reduce wrinkles on the skin.

Whereas bracing layer 362 is shown as planar and “static”, bracing layer 362 can, in accordance with an embodiment of the present invention, be a surface of a roller that is rolled over wrinkle foil 352 or a cylindrical surface that is rocked back and fourth over wrinkle foil 352. In some embodiments of the present invention, bracing layer 362 is a layer formed to match a desired shape of the body region being treated to ameliorate wrinkling. For example, if skin 356 is a region of skin on a person's forehead, bracing layer 362 is optionally formed to match the shape of the person's forehead in the region of skin 356.

In some embodiments of the present invention, a “matching” bracing layer 362 for a body region is formed by acquiring a 3D image of the surface of the region and forming the bracing layer responsive to the 3D image, using methods known in the art. The 3D image may be acquired, for example, by imaging the region with one of various different 3D cameras or scanners known in the art. The matching bracing layer 362 is of course formed without wrinkles that might be imaged in the acquired 3D image and the negative of the bracing layer is substantially a wrinkle free, “ideal” surface having a smoothness and shape that are aspired for the skin covering the body region.

In some embodiments of the present invention, an impression of the body region is made using an appropriate molding material, such as a non-toxic room temperature vulcanizing (RTV) rubber. Wrinkles in the impression are then removed and the matching bracing layer is formed from the impression using methods known in the art.

In accordance with some embodiments of the present invention, bracing layer 362 comprises a layer of piezoelectric material and a power supply for exciting ultrasound vibrations in the piezoelectric layer. The ultrasound vibrations radiate ultrasound into tissue in the region of skin being treated with bracing layer 362 and wrinkle foil 352. The piezoelectric layer and power supply are, optionally, integrated with bracing layer 362 in a manner similar to the way in which a piezoelectric layer is integrated with dielectric layer 222 shown in FIG. 14 and discussed below.

In some embodiments of the present invention, the piezoelectric layer is used not only to radiate ultrasound into tissue in the region of skin 356 but also to sense reflections of the transmitted ultrasound from regions in the skin and tissue underlying the skin. The reflections are processed by a suitable controller using methods known in the art to determine resonant frequencies of vibration of the skin and underlying tissue. In accordance with an embodiment of the present invention, the controller controls the piezoelectric layer to transmit ultrasound having a frequency component that is substantially equal to a resonant vibration frequency of skin 356 and/or tissue underlying the skin. In some embodiments of the present invention, for resonant frequencies of vibration that are substantially different form a convenient ultrasound frequency provide by the piezoelectric layer, the controller modulates ultrasound radiated by the piezoelectric layer with a suitable modulation function having a frequency substantially equal to a resonant vibration frequency. Stimulation of skin 356 and/or tissue underlying the skin at a resonant vibrator frequency, provides for efficient transfer of mechanical energy from the piezoelectric layer to the skin and/or underlying tissue and thereby for efficient mechanical stimulation of the skin and or underlying tissue.

In some embodiments of the present invention, bracing layer 362 comprises a heating element for heating treated skin. In addition, in some embodiments of the present invention, bracing layer 362 also includes a cooling element for cooling treated skin. In some embodiments of the present invention, bracing layer 362 comprises a Peltier thermocouple that can function as both a heating element and a cooling element. A region of skin being treated with wrinkle foil 352 and bracing layer 362 to reduce wrinkling, in accordance with an embodiment of the present invention, is alternately heated and cooled.

Heating softens “bent” collagen fibers in the region of skin being treated that provide structure to wrinkles in the region of skin. When the skin is flattened to the bracing layer and wrinkles are ironed out, in accordance with an embodiment of the present invention, softened collagen fibers in the wrinkles have a tendency to straighten. Ultrasound vibration during the heating and ironing abets the straightening of the collagen fibers. During cooling, the straightened collagen fibers have a tendency to “set” and remember their straightened configuration. As a result wrinkling of the skin tends to be lessened. (Whereas a Peltier thermocouple has been discussed with reference to bracing layer 362, it should be noted that Peltier thermocouples can be coupled to a skin-gripper used in a massager or other device described above for heating and cooling skin being treated using the skin-gripper, in accordance with embodiments of the present invention.)

In some embodiments of the present invention, vacuum is used to flatten a magnetic wrinkle foil attached to the skin to a bracing layer. Once the wrinkle foil is flattened to the bracing layer, magnetic forces between the wrinkle foil and the bracing layer maintain the wrinkle foil in contact with the bracing layer. In some embodiments of the present invention, a wrinkle foil is not formed from a magnetized material and vacuum is used without magnetic forces to flatten a wrinkle foil to a bracing layer and maintain the wrinkle foil flattened to the bracing layer.

FIG. 8C schematically shows a cross sectional view of a wrinkle foil 400 being used to treat wrinkles 402 in skin 404 of a body region 405 and a bracing layer 406 to which the wrinkle foil is flattened using vacuum. By way of example, it is assumed that wrinkle foil 400 is not magnetic and that the bracing layer is shaped to match the contours of body region 405.

As in the case of magnetic wrinkle foil 352, wrinkle foil 400 is kneaded into the skin so that a surface 408 of the foil is in contiguous contact with furrows 410 of wrinkles 402. In some embodiments of the present invention, an adhesive (not shown) adheres surface 408 of foil 400 to furrows 410 of wrinkles 402. In some embodiments of the present invention electrostatic forces are used to adhere wrinkle foil 400 to wrinkles 402. For example, wrinkle foil 400 may be formed from polyester, parafilm, Teflon or silicon. These materials can be charged and will “hold” a charge once charged so that wrinkle foil 400 can be made to adhere electrostatically to wrinkles 402. Wrinkle foil 400 may be charged by contacting the wrinkle foil with an electrode (not shown) connected to a suitable power supply or by frictional contact with a suitable electrostatic charger. By way of another example, wrinkle foil 400 may comprise a flexible conducting layer bonded to a flexible dielectric. The wrinkle foil is adhered to the skin by placing the dielectric side of the wrinkle foil on the skin and charging the conducting layer with respect to the skin using a suitable power source.

In some embodiments of the present invention, foil 400 is formed from a piezoelectric material such as polyvidelene deflourethane (PVDF), which when deformed by kneading generates a charge on surface 408. Once the charge is generated, induced charges in skin 404 cause wrinkle foil 400 to stick to furrows 410 of wrinkles 402.

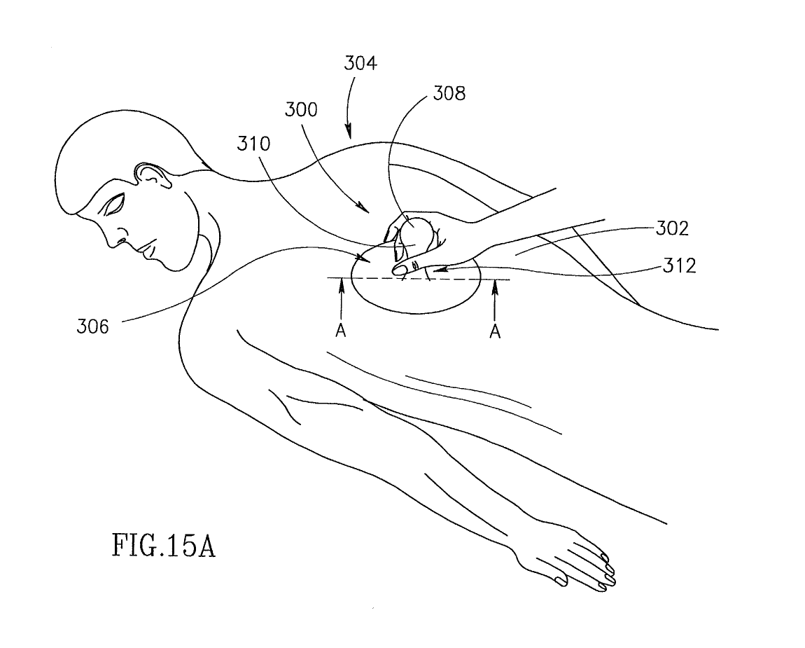

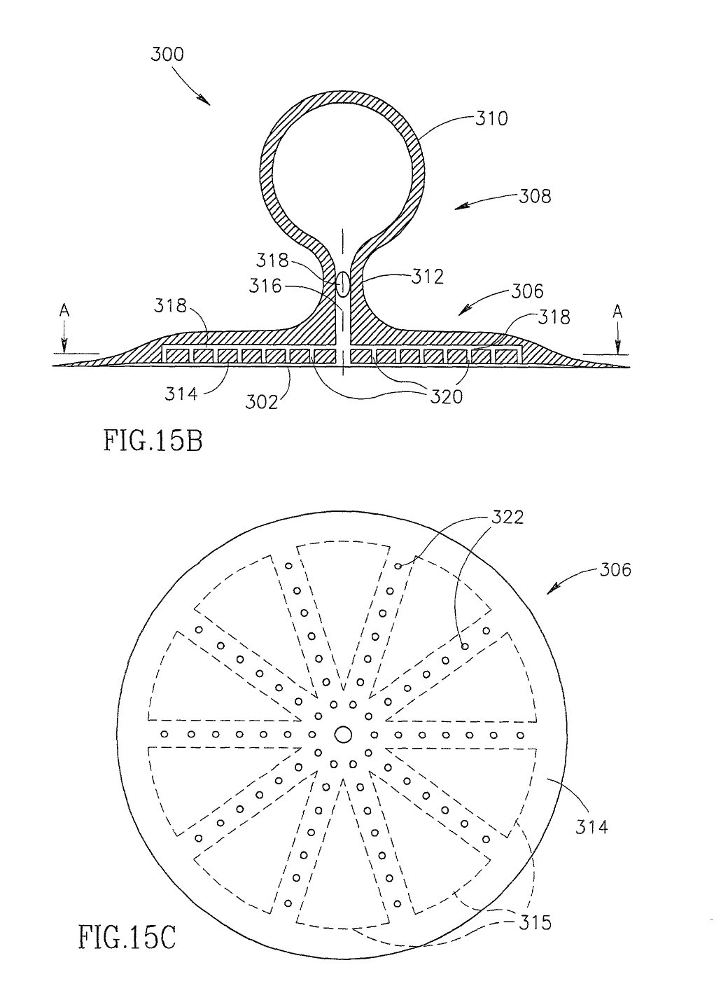

In some embodiments of the present invention, as is shown in FIG. 8C, bracing layer 406 is formed with a network of holes 420 and channels 422 connected to a main channel 426. Main channel 426 is connected to a vacuum pump (not shown) which aspirates air through the main channel and thereby through the network of channels 422 and holes 420. As air is aspirated through channels 422 and holes 420, air is evacuated from spaces 424 between the bracing layer and wrinkle foil 400 that occur at locations of wrinkles 402 into which the foil is kneaded and wrinkles 402 flatten to bracing layer 406. In some embodiments of the present invention, holes and channels in bracing layer 406 are formed in a configuration similar to that shown below in FIG. 15A for vacuum massager 300. In some embodiments of the present invention, bracing layer 406 is formed from a porous material. Air is aspirated through pores in the material, using methods known in the art, to evacuate air from spaces 424.

In some embodiments of the present invention, air is constantly or periodically aspirated by the vacuum pump through main channel 426 to maintain vacuum between wrinkle foil 400 and bracing layer 406. In some embodiments of the present invention, once wrinkle foil 400 is flattened to bracing layer 406 main channel 426 is sealed to maintain vacuum between wrinkle foil 400 and bracing layer 406. In some embodiments of the present invention, a bracing layer is used without a wrinkle foil and skin being treated for wrinkles, in accordance with an embodiment of the present invention, is flattened directly to the bracing layer by a vacuum.

Optionally, channels and holes, or pores, in a bracing layer through which air between the skin and the bracing layer is aspirated are “dusted” with a layer of small particles 428 (shown greatly exaggerated for convenience) that absorb water vapor and/or other gases that are released through the skin. Particles 428, which may for example be formed from silica and/or carbon, adhere to surfaces of the channels and pores as a result of electrostatic forces between the particles and the surfaces. By absorbing water vapor and other gaseous effluents released through the skin, particles 428 help to maintain vacuum between the bracing layer and the skin and thereby contact of the skin with the bracing layer. In addition, as a result of the absorption of the gaseous effluents, particles 428 aid in reducing buildup and “trapping” of the effluents on the surface of the skin. The effluents can be harmful to skin health and as a result, particles 428 aid in maintaining health and vigor of the skin.

In FIG. 8C bracing layer 406 is schematically shown with gas absorbing particles 428 adhering to walls of channels 422 and holes 420 of the bracing layer. In some embodiments of the present invention, wrinkle foils are impregnated with gas absorbing particles, which stick to pores and perforations in the wrinkle foil. The gas absorbing particles in the wrinkle foil work similarly to the gas absorbing particles in the bracing layer to remove gaseous effluents released through the skin.

In some embodiments of the present invention, in which a bracing layer is used without a wrinkle foil to treat wrinkles in a region of skin the bracing layer is formed from a suitable porous material or formed with a suitable “lacing” of channels and holes. Air is evacuated between the skin region and the bracing layer through the bracing layer by an appropriate vacuum pump and the skin region is flattened directly to the bracing layer by vacuum.

The inventors have found that when a foil is not used a vacuum used to draw out and flatten wrinkles to a bracing layer should preferably be a partial vacuum for which pressure between the skin and the bracing layer does decrease below about 0.75 atmospheres. Pressures substantially less than about 0.75 atmospheres can cause hemorrhaging of blood vessels in the skin and damage the skin. Whereas optimum pressures for partial vacuums used to flatten wrinkles to a bracing layer can depend upon characteristics of the skin being treated, preferably, pressure of a partial vacuum used to flatten wrinkles to a bracing layer is maintained between 0.95 and 0.80 atmospheres. More preferably, the pressure is maintained between 0.90 and 0.80 atmospheres. Most preferably, the pressure is substantially equal to 0.85 atmospheres.

In some embodiments of the present invention, a “wrinkle” foil used for treatment of wrinkles, in accordance with an embodiment of the present invention, is used without a bracing layer. The foil is formed from a pliable viscoelastic or elastoplastic material that returns completely or partially to its original shape when stress that deforms the material is removed. After the wrinkle foil is kneaded or pressed into a furrow of a wrinkle and adhered to the furrow, the foil returns to its original shape exercising forces on the furrow that draws the furrow outward to flatten the wrinkle. The wrinkle foil is adhered to the skin using any of the methods used to adhere wrinkle foils to skin described above. A wrinkle foil with suitable characteristics for use in treating wrinkles without use of a bracing layer, in accordance with an embodiment of the present invention, can be formed, for example, from a suitable biopolymer.

FIG. 8D shows a wrinkle foil 430 that is used to treat wrinkles without use of a bracing layer, in accordance with an embodiment of the present invention. Wrinkle foil 430 is shown, by way of example, as a ribbon shaped foil being dispensed from a roll 432 and being pressed into a furrow 434 of a wrinkle 436, optionally, by an application wheel 438. In FIG. 8D wrinkle foil 430 is assumed, by way of example, to be adhered to the skin in furrow 434 and regions in the neighborhood of wrinkle 436 by electrostatic forces.

In order to charge wrinkle foil 430, in some embodiments of the present invention, application wheel 438 is connected to a suitable power supply (not shown) so that as the application wheel presses wrinkle foil 430, the wrinkle foil is charged and adheres to the skin. In some embodiments of the present invention, application wheel 438 slides on wrinkle foil 430 as the wrinkle foil is pressed to wrinkle 436 and wrinkle foil 430 is charged by frictional contact between the application wheel and the wrinkle foil.

In some embodiments of the present invention wrinkle foil 430 is formed from a dielectric layer (not shown) which is placed in contact with the skin and a conducting layer (not shown). Wrinkle foil 430 is charged by connecting the conducting layer to a suitable power supply, which may, for example, be a small battery that can easily be carried or adhered to the skin.

To aid in “molding” a wrinkle foil to a wrinkle, a ribbon shaped wrinkle foil similar to wrinkle foil 430 may be formed with scalloped edges. FIG. 8E shows a ribbon shaped wrinkle foil 442 having scalloped edges 444, in accordance with an embodiment of the present invention. Scalloping of the edges of a wrinkle foil reduces wrinkling of the foil along the foil edges when the foil is deformed to the shape of a wrinkle into which it is being kneaded or pressed.

It is to be noted that a wrinkle foil suitable for use without a bracing layer requires sufficient elasticity so that after it is deformed and molded into wrinkles being treated, the wrinkle foil relaxes and substantially recovers its original dimensions and flattens thereby the wrinkles. However, the wrinkle foil does not require equal elasticity in all directions. It can be advantageous for the wrinkle foil to have relatively increased rigidity with respect to bending along a direction perpendicular to the length of a wrinkle into which the wrinkle foil is pressed relative to rigidity of the foil in other directions. Increased rigidity perpendicular to the length of the wrinkle aids in drawing out furrows of the wrinkles and flattening the wrinkles after the foil is pressed into the wrinkle.

Therefore, in some wrinkle foils in accordance with an embodiment of the present invention, the foil is formed using methods known in the art with an increased rigidity along one direction and reduced rigidity in other directions. For example, wrinkle foil 430 shown in FIG. 8D, optionally, has enhanced rigidity perpendicular to its length, in a direction indicated by double arrowhead line 446. Enhanced rigidity in the indicated direction may be provided by forming wrinkle foil 430 with narrow “thickened” ribs parallel to arrowhead line 446 or by treating the wrinkle foil material to increase stiffness of bands of the material in the direction of arrowhead line 446. For example, a wrinkle foil formed from a suitable polymer substrate might be impregnated by a photopolymer emulsion which is then stiffened in an appropriate pattern by exposure to ultraviolet light.

As in the case of wrinkle foils that are used with a bracing layer, wrinkle foils that are not used with a bracing layer are, optionally, perforated and/or formed from a porous material to allow skin to which they are applied to breath. Optionally, perforations and or pores in the wrinkle layer are suffused with gas absorbing particles that absorb water vapor and other gases released through the skin and reduce trapping of the gases on the surface of the skin.

In some embodiments of the present invention, a wrinkle foil whether used with or without a bracing layer, comprises a piezoelectric layer. A power supply coupled to the piezoelectric layer excites ultrasound vibrations in the layer that penetrate the skin and aid in reducing wrinkling.

It should be noted that trapping of gases released through the skin often occurs on regions of skin that are bandaged to protect a wound in the skin region. Deleterious effects of the trapping, such as for example discoloring, wrinkling and/or loss of resilience, in skin that is bandaged for extended periods of time, are common. In accordance with an embodiment of the present invention, a bandage used to cover a region of skin is formed with perforations or pores and the perforations and or pores are suffused with gas absorbing particles. As in the case of wrinkle foils, the gas absorbing particles in the bandage aid in reducing trapping on the surface of the skin, gases that are released through the skin. The particles reduce thereby the deleterious effects of the gases on the skin.

Whereas wrinkle foil 430 is shown in FIG. 8D being pressed into wrinkle 436 by application wheel 438, a wrinkle foil can, in accordance with an embodiment of the present invention, be pressed to a wrinkle using an applicator stick. If the wrinkle foil is adhered to the skin by electrostatic forces, optionally, the applicator stick functions also to charge the wrinkle foil.

FIG. 8F shows a wrinkle foil 450 similar to wrinkle foil 430 being applied by a woman to a wrinkle (not shown) in her forehead using an applicator stick 452 having a tip 454 that is pressed to the wrinkle foil. Applicator stick 452 comprises a power supply (not shown) that generates a potential difference between the woman's hand and tip 454, which potential difference charges wrinkle foil 450 and adheres the wrinkle foil to the woman's skin, and in particular to skin in the furrow of the wrinkle.

In some embodiments of the present invention, positive air pressure is used to press a wrinkle foil into wrinkles on a region of skin being treated to reduce wrinkling. The wrinkle foil is attached to a bracing layer so that air can be pumped between the wrinkle foil and the bracing layer so as to distend the wrinkle foil away from the bracing layer without detaching the wrinkle foil from the bracing layer. When not distended by air pressure, the wrinkle foil lies flat on the bracing layer. The bracing layer is formed to resist bending along at least one “stiff” direction and when used, the bracing layer is positioned on the skin with the wrinkle foil in contact with the skin and with the stiff direction substantially perpendicular to the length of the wrinkle. The wrinkle foil is adhered to skin that it contacts using any of the methods noted above. Thus positioned on a wrinkle, the bracing layer is held pressed to the wrinkle and air is pumped between the wrinkle foil and the bracing layer so as to distend the wrinkle foil and force the wrinkle foil into the furrow of the wrinkle. Air between the wrinkle foil and the bracing layer is then evacuated or allowed to escape so that the wrinkle foil recovers its shape to lie flat on the bracing layer and flatten thereby the wrinkle.

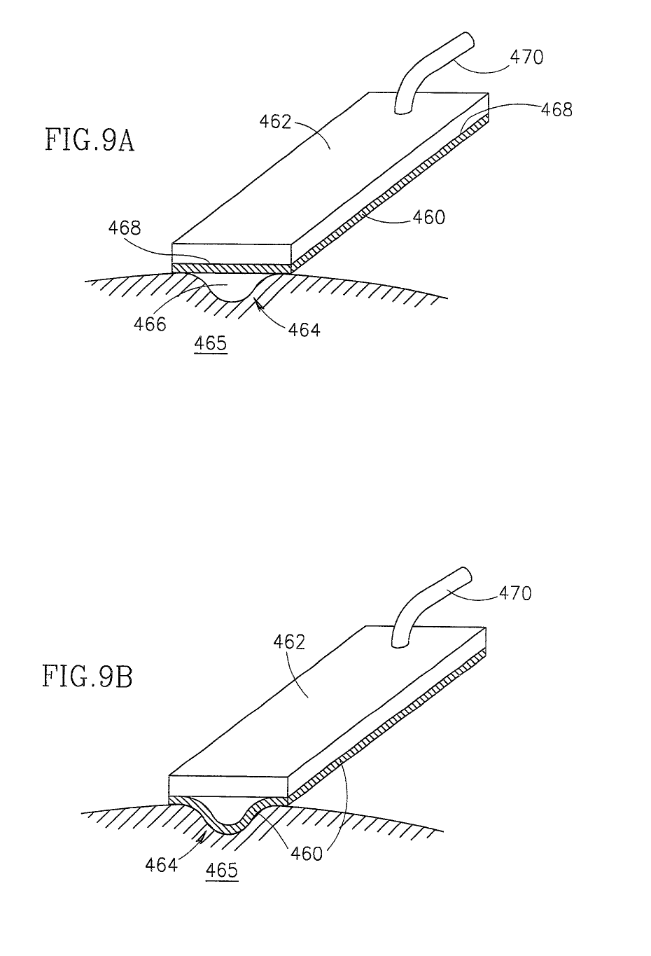

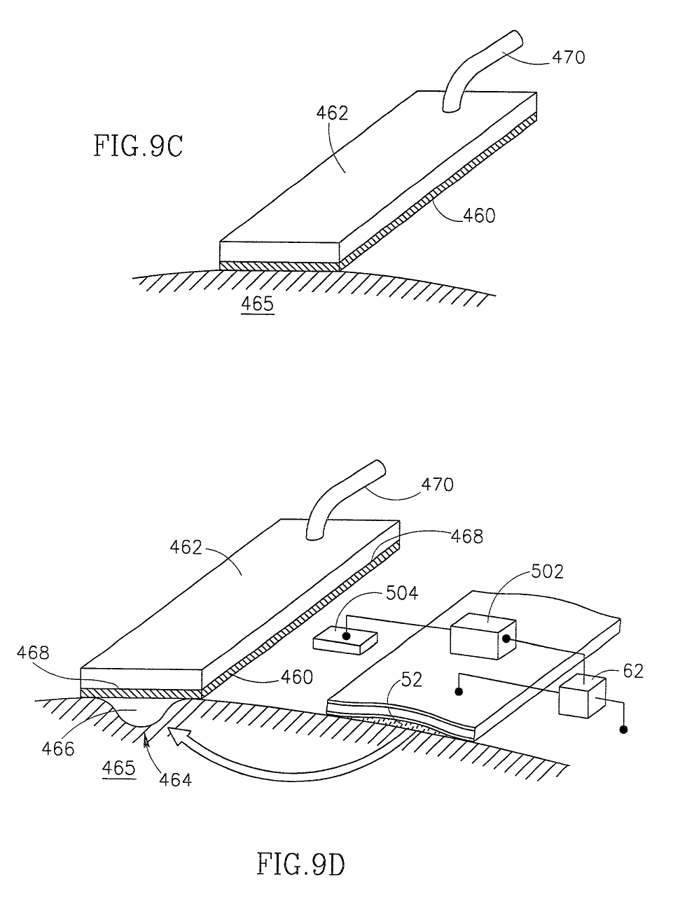

FIGS. 9A-9C schematically show a cross section and partial perspective view of a wrinkle foil 460 attached to a bracing layer 462 being used to treat a wrinkle 464 having a furrow 466 in skin 465, in accordance with an embodiment of the present invention.

By way of example, bracing layer 462 is a thin rectangular shaped ribbon resiliently resistant to bending across the width of the ribbon, formed of a suitable material such as plastic or Teflon. Wrinkle foil 460 is sealed to bracing layer 462 substantially only along edges 468 of the bracing layer. An inlet tube 470 allows air to be forced between wrinkle foil 460 and bracing layer 462 by a suitable air pump (not shown) attached to the inlet tube.

In FIG. 9A wrinkle foil 460 is shown lying flat on its bracing layer 462 and positioned over wrinkle 464. In FIG. 9B air is forced between wrinkle foil 460 and bracing layer 462 by the pump, causing the wrinkle foil to distend and contact skin in furrow 466 of wrinkle 464. In FIG. 9C air is allowed to escaped from between wrinkle foil 460 and bracing layer 462 and wrinkle foil 460 recovers its shape to flatten wrinkle 466 to bracing layer 462.

In some embodiments of the present invention, a device for treating wrinkles in a region of skin is used with a perfusion skin-gripper that perfuses a substance beneficial for ameliorating wrinkles into the body transdermally.

By way of example of use of a wrinkle treating device in combination with a perfusion skin-gripper, FIG. 9D shows a schematic cross section and partial perspective view of wrinkle foil 460 shown in FIGS. 9A-9C being used with perfusion skin-gripper 500, shown in FIG. 4B, to treat wrinkle 464. During the process of treating wrinkle 464, perfusion skin-gripper 500 perfuses a substance 52 beneficial in the treatment of wrinkles through skin 465. As a result of stimulation of skin 465 in the region of wrinkle 464, when air is aspirated from furrow 466 of wrinkle 464, migration of substance 52 introduced into skin 465 by perfusion skin-gripper 500 towards wrinkle 464 is enhanced. Enhanced migration of substance 52 towards wrinkle 464 is schematically represented by block arrow 510.

FIG. 1OA schematically shows a “pattern massager” 120 comprising a flexible skin-gripper 122 used to massage the skin. Skin-gripper 122 comprises a flexible insulating substrate 124 and a flexible dielectric layer 126 having a gripping surface 127. In some embodiments of the present invention a conducting layer 128 sandwiched between layers 124 and 126 optionally comprises a plurality of relatively thin parallel strip conductors 130 shown with ghost lines. Each strip conductor 130 is electrified independently of other strip conductors 130 by a power supply (not shown) that is connected to strip electrodes 130 by an appropriate switching circuit (not shown). The power supply is grounded to the skin through a grounding conductor attached to the skin using methods known in the art or by using a skin-gripper in accordance with an embodiment of the present invention that presses the conductor to the skin. In some embodiments of the present invention, the grounding conductor is pressed to the skin by the action of strip conductors 130 that are electrified to press the grounding conductor to the skin during the time that pattern massager 120 is in operation.

To massage the skin, skin-gripper 122 is placed on the skin and the power supply and switching circuit are controlled to apply gripping voltages to strip-conductors 130 in varying spatial and/or temporal patterns. As different strip conductors 130 are electrified with gripping voltages, different areas of the skin in contact with skin-gripper 122 are gripped and exercised.

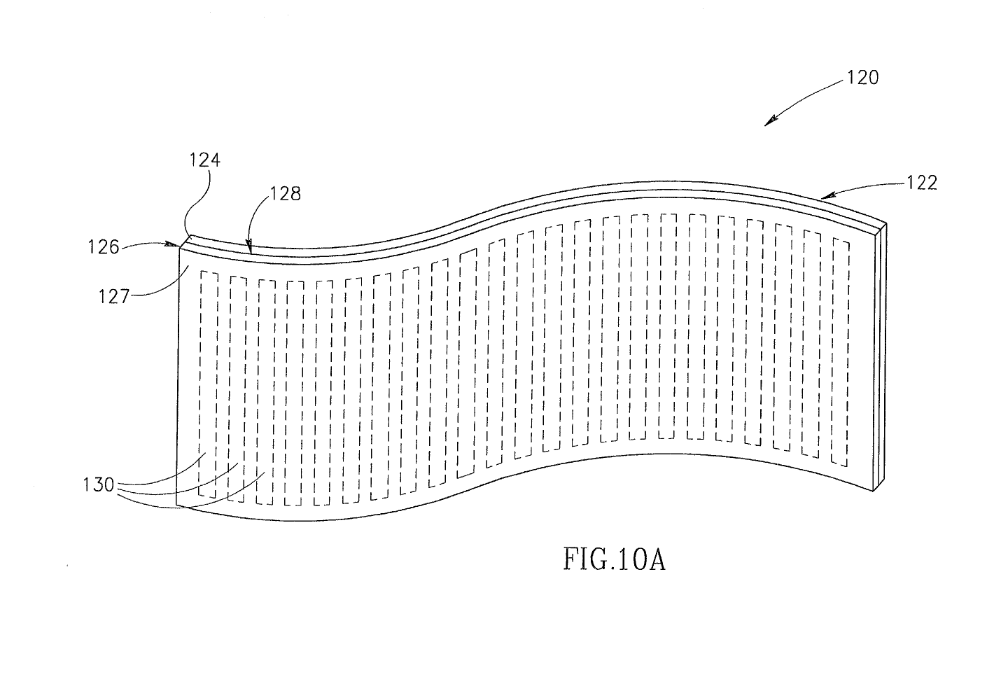

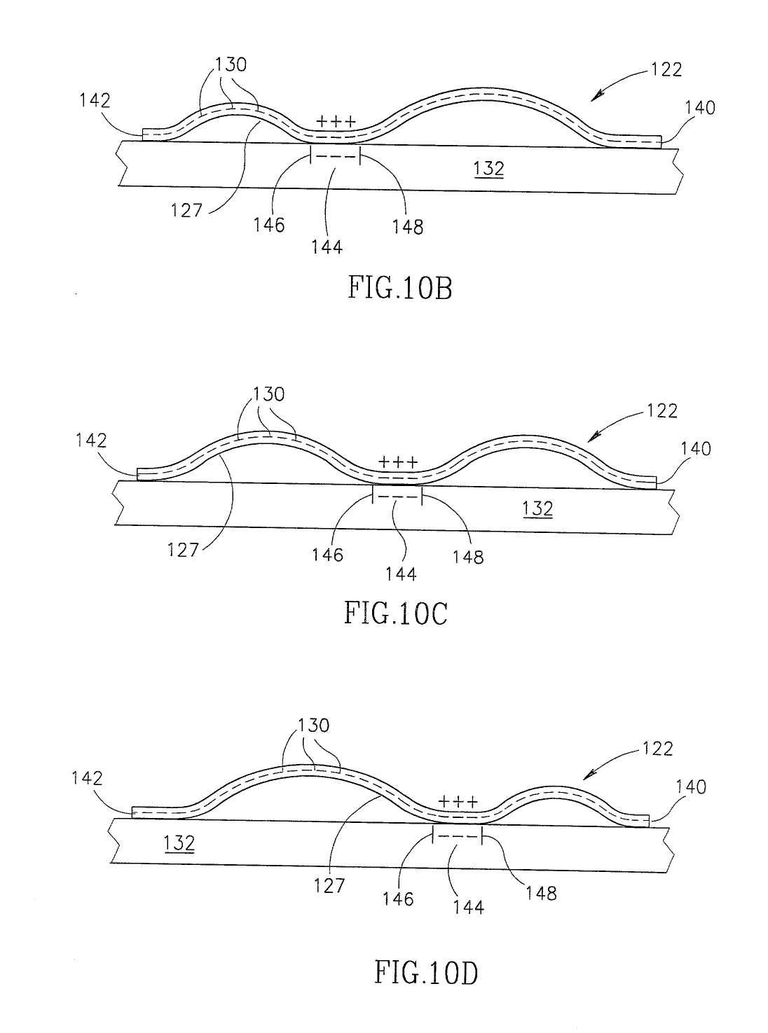

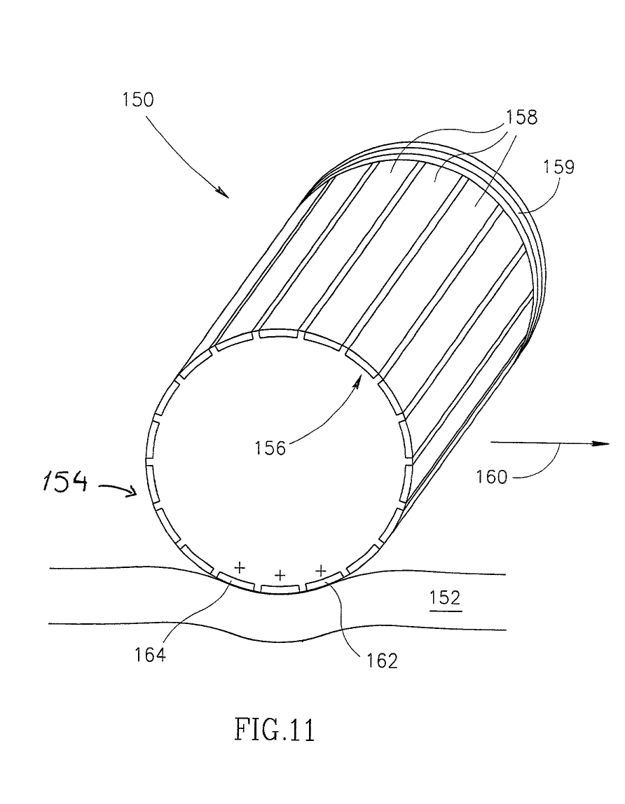

FIGS. 10B-10D schematically illustrate a method by which skin-gripper 122 massages a region of a person's skin 132, in accordance with an embodiment of the present invention. In FIGS. 10B-10D skin-gripper 122 and skin 132 are shown in a cross section view that is perpendicular to the lengths of strip conductors 130.

Skin-gripper 122 is optionally attached and “anchored” in place to skin 132 by placing ends 140 and 142 of skin-gripper 122 in contact with skin 132 and controlling the power supply and switching circuit to apply a gripping voltage to at least one strip conductor 130 at each of ends 140 and 142. Preferably the distance between ends 140 and 142 on skin 132 is less than the length of skin-gripper 122. Therefore, at any one time, not all of gripping surface 127 can be in contact with skin 132.

To massage skin 132, in accordance with an embodiment of the present invention, the power supply and switching circuit are controlled, by way of example, to electrify at any one time, only one or a small group of adjacent strip conductors 130 located between ends 140 and 142 of skin-gripper 122. In FIGS. 10B-10D, by way of example, a group of three conductors 130 between ends 140 and 142 are electrified by being charged positively with respect to skin 132. Electrified strip conductors 130 are indicated by +signs over the conductors. As a result, at any one time, skin-gripper 122 contacts and grips skin 132 only along a strip 144 of skin 132 opposite electrified conductors 130. Strip 144 is shown between witness lines 146 and 148 and has a negative induced charge indicated by minus signs.

By electrifying different strip conductors 130 so as to move the position of the group of electrified conductors 130 right or left, the position of strip 144 that is gripped by skin-gripper 122, is moved right or left. FIGS. 10B-10D schematically show, by way of example, the position of strip 144 at respectively later times as electrified strip conductors 130 are “moved” to the right along skin-gripper 122, in accordance with an embodiment of the present invention. As a result of the motion of strip 144 a travelling wave appears to be moving along skin-gripper 122 to the right. The motion of the gripped area of skin massages the skin. As with other massagers described above, in some embodiments of the present invention, microcurrents flow to the skin at points of contact between skin-gripper 122 and skin 132.

Whereas FIGS. 10B-10D show skin-gripper 122 contacting skin 132 only along one strip between ends 140 and 142, in some embodiments of the invention skin-gripper 122 contacts skin 132 along more than one strip between ends 140 and 142. Furthermore, flexible skin-grippers, in accordance with embodiments of the present invention, may comprise arrays of conductors having conductor shapes and configurations different from the configuration of conductors comprised in wave massager 122, and these different arrays can be advantageous. For example, because conductors 130 are arranged in a one dimensional array, skin-gripper 122 can provide a gripping area that moves back and forth only along a single direction, the direction perpendicular to the lengths of strip conductors 130. (The conductor array in skin-gripper 122 is one dimensional because the array is formed by positioning strip conductors 130 parallel to each other along a single direction.) In some pattern massagers, in accordance with embodiments of the present invention, conductors in a conducting layer of a skin-gripper comprised in the pattern massager are arranged in a two dimensional array. For example a pattern massager, in accordance with an embodiment of the present invention, might have a conductor array similar to the two dimensional conductor array of stretch massager 90 shown in FIG. 7B. With a two-dimensional conductor array a gripped area can be moved in two directions over a region of skin that is in contact with the pattern massager.

FIG. 11 schematically shows a perspective cross section view of a motile massager 150 moving over a person's skin 152, in accordance with an embodiment of the present invention. In some embodiments of the present invention, motile massager 150 comprises a skin-gripper 154 having a circularly cylindrical gripping surface 156 formed from a plurality of identical parallel strip conductors 158 covered with a dielectric layer (not shown). A power supply (not shown) and switching circuit (not shown) electrify strip conductors 158 independently of each other. Optionally, two ground strips 159 (only one of which is shown) that completely band gripping surface 156, one at each end of gripping surface 156, are used for grounding the power supply to skin 152.

At any one time only a portion of gripping surface 156 contacts skin 152 and strip conductors 158 in the portion are charged to a gripping voltage to grip skin 152 and hold skin-gripper 154 to the skin. In FIG. 11 strip conductors 158 that are charged to grip skin 152 are indicated, by way of example, as being charged positive with respect to skin 152. In some embodiments of the present invention strip conductors 158 are charged negatively with respect to skin 152.

Skin-gripper 154 rolls over skin 152 by itself. To accomplish this the power supply is switched by the switching circuit to repeatedly, substantially simultaneously, discharge a strip conductor 158 at one edge of a group of charged strip conductors 158 that are gripping skin 152 and charge a strip conductor 158 at the opposite edge of the group of strip conductors 158. As a result of the charging and discharging of strip conductors 158 in this manner, skin-gripper 154 rolls in the direction of the edge of the group of strip conductors 158 along which strip conductors 158 are being charged. For example, in FIG. 11, skin-gripper 154 will roll in the direction of arrow 160 if the strip conductor 158 labeled with numeral 162 is charged and the strip conductor 158 labeled with numeral 164 is discharged.

In operation, after skin-gripper 154 rolls along the skin for a desired distance, the power supply and switching circuit “reverse direction” so that skin-gripper 154 rolls back and forth over a desired region of skin. The rolling motion of skin-gripper 154 massages skin 152. In some embodiments of the present invention, skin-gripper 154 is constructed so that electrical micro-currents flow between strip conductors 158 that contact skin 152 and skin 152 in a similar manner as described above and to provide similar beneficial effects.

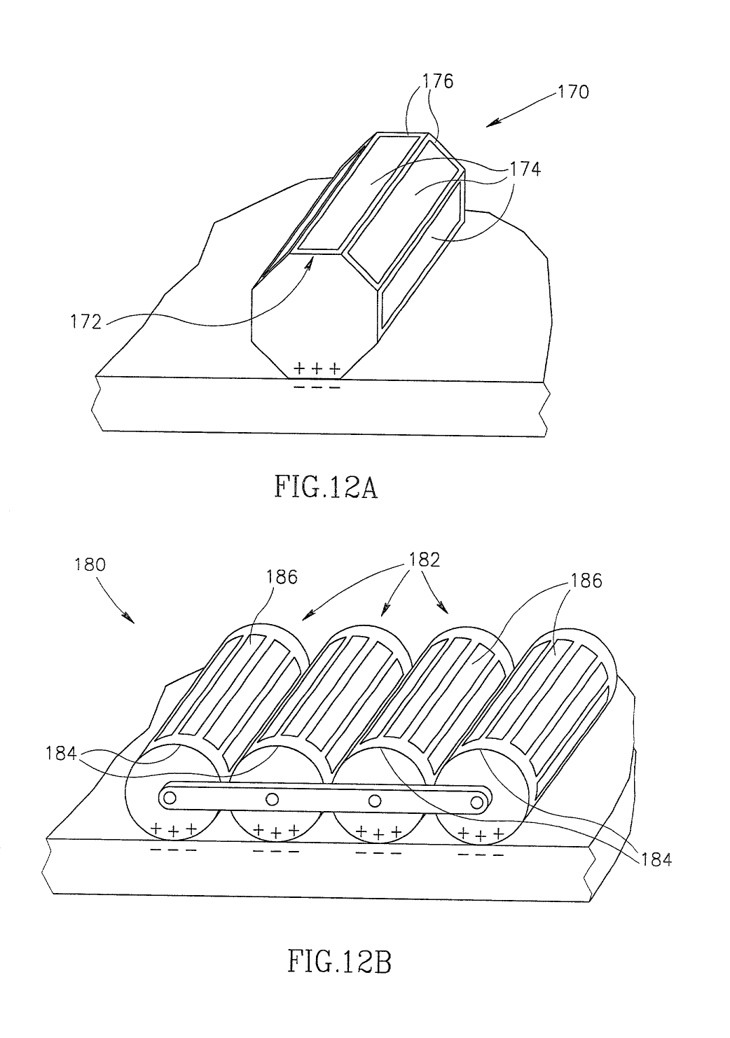

Geometries for motile massagers, in accordance with embodiments of the present invention, that are different from the geometry of motile massager 150 are possible and can be advantageous. For example, a motile massager 170 in accordance with an embodiment of the present invention may comprise a cylindrical gripping surface 172 defined by a directrix that is an equilateral polygon as shown in FIG. 12A. Strip conductors 174 are located on panels 176 of polygonal cylindrical surface 172 and are electrified by an appropriate power supply (not shown) to rotate gripping surface 172 in similar manner to the way strip conductors 158 of skin-gripper 154 shown in FIG. 11 are electrified to rotate skin-gripper 154. Or, by way of another example, a motile massager 180, as shown in FIG. 12B, may comprise a plurality of skin-grippers 182 having circularly cylindrical gripping surfaces 184 and strip conductors 186.

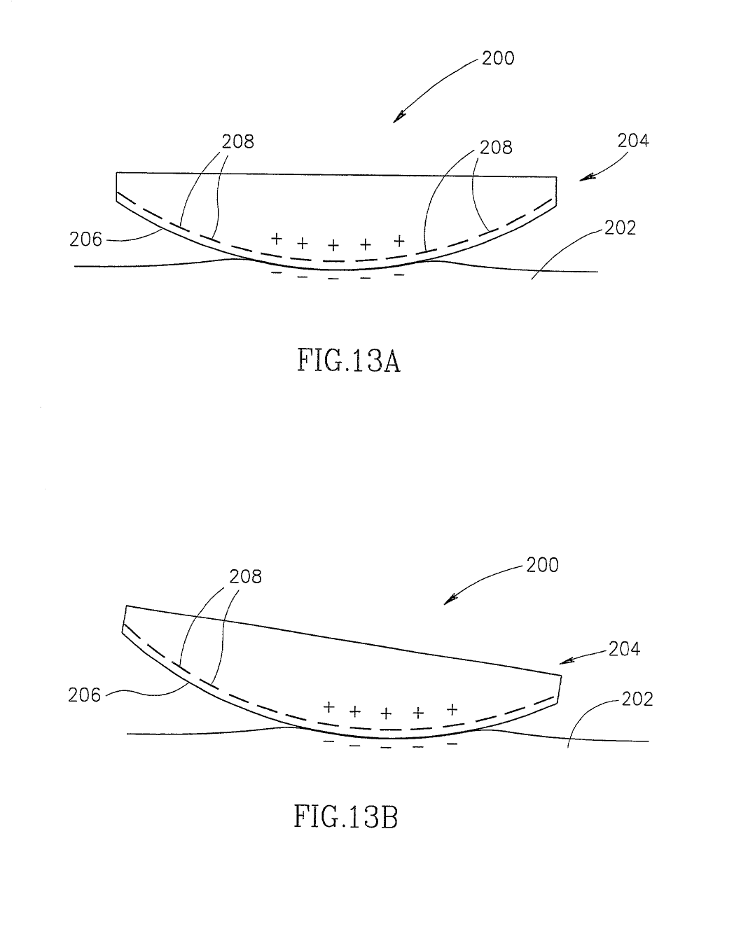

FIGS. 13A and 13B schematically show a cross section view of two positions of a motile massager 200 that rocks by itself back and forth over a persons skin 202. Motile massager 200 comprises a skin-gripper 204 having a cylindrical gripping surface 206 comprising strip conductors 208 (only the edges of which are shown in FIGS. 13A and 13B) similar to strip conductors 158 of skin-gripper 154 shown in FIG. 11. As in the case of skin-gripper 154, at any one time only a portion of gripping surface 206 contacts skin 202 and only some strip conductors 208 are charged to attract and grip an area of skin 202. And in similar manner to the way in which skin-gripper 154 is rolled along a person's skin, skin-gripper 204 is rocked back and forth over skin 202 by appropriately charging and discharging strip conductors along the edges of a group of strip conductors gripping skin 202.

FIG. 14 schematically shows a massager 210, similar to massager 64 shown in FIG. 5, for treating a region of skin 212 with ultrasound radiation as the skin is massaged, in accordance with an embodiment of the present invention.

Massager 210 comprises a skin-gripper 211 having a substrate body 214 to which is bonded a piezoelectric layer 216 sandwiched between two conducting layers 218 and 220. Conducting layer 220 is covered with a thin dielectric layer 222. Surface 224 of dielectric layer 222 is a gripping surface of skin-gripper 212.

A high frequency power supply 226 generates a high frequency AC voltage between conducting layers 218 and 220 to excite ultrasound vibrations in piezoelectric layer 216. A DC power supply 228 generates a potential difference, i.e. a gripping potential, between conducting layer 220 and skin 212. Optionally, contact between DC power supply 228 and skin 212 is made through a conducting strip (not shown) located on gripping surface 224 or through an electrode (not shown) attached and held to skin 212 by a skin-gripper powered by DC power supply 228. When massager 210 is rocked back and forth on skin 212 ultrasound waves are radiated into skin 212 and into tissue below skin 212 at the area of contact between gripping surface 224 and skin 212.

Whereas FIG. 14 shows a piezoelectric layer integrated with a massager having a relatively non-flexible gripping surface, piezoelectric transducers for providing ultrasound stimulation of a massaged region may be incorporated into flexible and stretchable massagers, in accordance with embodiments of the present invention. For example, flexible pattern massager 120 shown in FIG. 10A may comprise a flexible piezoelectric layer and stretch massager 90 shown in FIG. 7B may be “seeded” with a plurality of piezoelectric transducers that do not interfere substantially with the “stretchability” of the massager.

FIG. 15A schematically shows a vacuum massager 300, in accordance with an embodiment of the present invention. Vacuum massager 300 uses a vacuum to treat the skin of a patient. In FIG. 15A vacuum massager 300 is shown being used to treat a region of skin 302 on the back of a patient 304.

Vacuum massager 300 comprises a skin-gripper 306, in the form of a flange that is connected to a vacuum pump 308. Optionally, skin-gripper 306 is flexible. Vacuum pump 308 is used to evacuate air and produce a partial vacuum between the gripping surface (which is in contact with skin 302 and not shown) of skin-gripper 306 and skin 302. Vacuum pump 308 may be a manually operated vacuum pump or an electrically operated vacuum pump. In FIGS. 13A and 13B vacuum pump 308 is shown, by way of example as a manually operated bulb type vacuum pump comprising a flexible bulb 310 connected to skin-gripper 306 by a “neck” 312. Vacuum pump 308 evacuates air between skin-gripper 306 and the patient's skin 302, optionally, through a central air channel (not shown) in neck 312 when bulb 310 is repeatedly squeezed and released.

In some embodiments of the present invention, the conducting layer of skin-gripper 306 comprises a plurality of flexible conductors (not shown). A power supply (not shown) electrifies conductors to grip the skin and generate static and/or electric fields to treat the skin and tissue beneath the skin. Some vacuum massagers, in accordance with embodiments of the present invention, comprise a piezoelectric layer for generating ultrasound waves to treat the skin and/or a heating element to treat the skin with heat.

FIG. 15B schematically shows vacuum massager 300 in cross section view taken through a line AA shown in FIG. 15A. Skin-gripper 306 comprises a gripping surface 314. Conductors in the conducting layer of skin-gripper 306, optionally, do not cross the plane of the cross section perspective of FIG. 15B and conductors in the conducting layer are not shown. Neck 312 of vacuum massager 300 comprises a central air channel 316. Central air channel 316 is preferably fitted with a one way valve 318 that permits air to flow only in a direction into bulb 310. Air channel 316 may extend through gripping surface 314 so that air is evacuated from between skin 302 and gripping surface 314 directly through central air channel 316. Optionally, central air channel 316 is connected to a plurality of manifold air channels 318, two of which are shown in the cross section view of FIG. 15B, that extend radially from central air channel 316. Each manifold channel 318 is preferably connected by a plurality of secondary air channels 320 that extend from the manifold channel through gripping surface 314 and through which air is aspirated.

FIG. 15C shows a view of gripping surface 314 of skin-gripper 306 that shows openings 322 in gripping surface 314 that are located at points where secondary air channels 320 extend through gripping surface 314. In some embodiments of the present invention, the conducting layer of skin-gripper 300 comprises triangular shaped conductors 315 that are located between the rows of openings 322. Conductors 315 are shown in ghost lines because they are located under gripping surface 314. Conductor configurations different from that shown in FIG. 15C, including configurations in which conductors intrude into spaces between secondary air channels 320, are possible and can be advantageous and these configurations will occur to a person of the art. Furthermore, configurations of air channels for evacuating air from between skin-gripper 306 and skin 302 other than that shown in FIGS. 15B and 15C, are possible and can be advantageous and will occur to a person of the art.

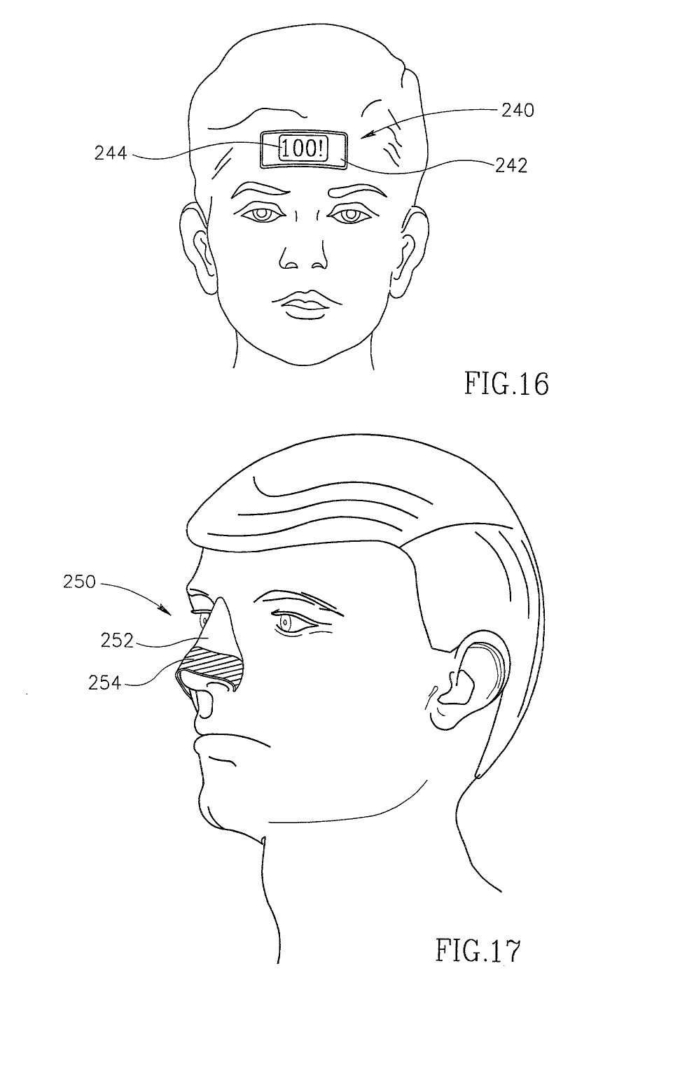

FIG. 16 schematically shows a thermometer 240 comprising a thin flexible skin-gripper 242, in accordance with an embodiment of the present invention, attached to a person's forehead to measure the person's temperature. Thermometer 240 comprises a heat-sensing element (not shown), such as for example a thin foil thermometer common in the art, which is held in place on the forehead by skin-gripper 240. An appropriate flexible flat panel display 244 is mounted to the skin-gripper to display a temperature reading. Whereas thermometer 244 is placed on the forehead it is obvious that it can be placed elsewhere on the body.

FIG. 17 schematically shows a “nose guard” 250 for protecting a person's nose from the sun's rays and preventing sunburn, in accordance with an embodiment of the present invention. Nose guard 250 comprises a flexible shade 252 mounted to a flexible skin-gripper 254. Skin-gripper 254 is shown as a shaded region, and grips the nose strongly in regions of the nose near the nostrils.

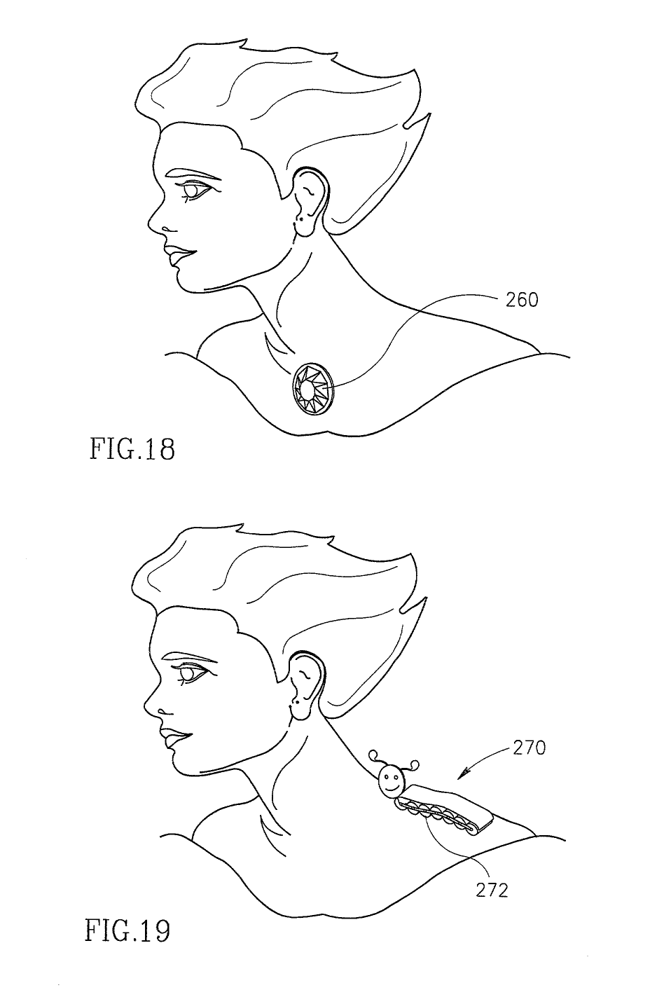

FIG. 18 schematically shows a decorative piece of jewelry 260 mounted on a skin-gripper (behind piece of jewelry 260 in FIG. 18) that holds the in place on a woman's neck, in accordance with an embodiment of the present invention.

FIG. 19 schematically shows a woman wearing a decorative “caterpillar” 270, which is shown in a partial cutaway view, that is formed on a “small” motile skin-gripper 272 so that caterpillar 270 moves along the woman's shoulder. Motile skin-gripper 272 may be similar in shape and construction to motile massager 180 shown in FIG. 12B. Motile skin-gripper 272 comprises a power supply and switching circuitry controlled to electrify strip conductors in motile skin-gripper 272 so that caterpillar 270 moves back and forth along the woman's neck.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A method for treating blemishes having furrows on a region of a person's skin comprising:
positioning a surface of a bracing body so that the surface contacts or is proximate to the region skin; and
attracting the region of skin to the surface so that blemishes in the region of skin are flattened to the surface.

2. A method according to claim 1 wherein attracting the region of skin comprises adhering a thin foil to the skin and attracting the thin foil to the surface of the bracing body.

3. A method according to claim 2 wherein adhering comprises kneading the foil so that it contacts and sticks to skin in furrows of blemishes in the region of skin.

4. A method according to claim 2 wherein adhering comprises forcing air between the surface of the bracing body and the foil so as to increase pressure between the bracing body surface and the foil and press thereby the foil into furrows of the blemishes.

5. A method according to claim 3 wherein adhering to the skin comprises adhering with an adhesive.

6. A method according to claim 3 wherein adhering to the skin comprises electrically charging the foil with respect to the skin so that electrostatic forces between the charged foil and induced charge in the skin adhere the foil to the skin.

7. A method according to claim 2 wherein the foil is a magnetized foil.

8. A method according to claim 7 wherein attracting comprises positioning a magnetized element in proximity to the magnetized foil so that magnetic forces between the element and the foil attract the foil to the surface of the bracing body.

9. A method according to claim 8 wherein the bracing body comprises the magnetized element.

10. A method according to claim 1 wherein the bracing body surface is a surface of a thin layer of dielectric material bonded to a conductor and attracting the skin comprises applying a voltage between the conductor and the skin.

11. A method according to claim 10 wherein attracting the region of skin comprises pressing the dielectric surface to the region of skin so that furrows of blemishes in the skin are flattened to and contact the dielectric surface.

12. A method according to claim 1 wherein attracting the region of skin comprises aspirating air from between the region of skin and the surface of the bracing body.

13. A method according to claim 1 and comprising shaping the surface of the bracing body so that the contour of the surface is substantially a negative of the contour of the region of skin exclusive of contour features resulting from the blemishes in the skin.

14. A method for treating blemishes having furrows on a region of a person's skin comprising:
placing on the region of skin a thin pliable foil that at least partially recovers its shape after being deformed when forces responsible for the deformation are removed;
applying forces that deform the foil so that it contacts skin in furrows of the blemish;
adhering the deformed foil to the blemish; and
removing the applied forces so that the foil at least partially recovers its undeformed shape.

15. A method according to claim 14 wherein adhering comprises using an adhesive.

16. A method according to claim 14 wherein adhering comprises electrically charging the foil with respect to the skin so that electrostatic forces between the charged foil and induced charge in the skin adhere the foil to the skin.

17. A method according to claim 14 and comprising heating the region of skin.

18. A method according to claim 14 and comprising cooling the region of skin.

19. A method according to claim 1 and comprising:
heating the skin to soften collagen fibers in the skin; and
subsequently cooling the skin while the blemishes remain flattened to the surface so that the collagen fibers retain a memory of their flattened configuration.

20. A method according to claim 1 and comprising mechanically stimulating the region of skin using ultrasound waves.

21. A method according to claim 20 wherein mechanically stimulating comprises stimulating the skin at a resonant frequency of vibration of the skin.

22. A method according to claim 21 and comprising determining a resonant frequency of vibration of the skin using ultrasound.

23. A method according to claim 1 and comprising stimulating the region of skin at a frequency of peristaltic waves of pores in the skin.

24. A method according to claim 23 wherein stimulating the region of skin at the peristaltic frequency of the pores comprises stimulating the skin electrically.

25. A method according to claim 23 wherein stimulating the region of skin at the peristaltic frequency comprises stimulating the skin mechanically.

26. A method according to claim 1 and comprising perfusing a substance beneficial for treating the blemish transdermally.

27. A method according to claim 26 wherein perfusing comprises:
sandwiching the substance between a region of skin and a surface of a conductor coated with a thin dielectric layer; and
applying a voltage between the conductor and the skin.

28. A method according to claim 27 and comprising controlling the voltage to control the rate at which the substance is perfused.

29. A method according to claim 28 and comprising modulating the voltage at a frequency of peristaltic waves of pores in the region of skin.

30. A method according to claim 1 wherein a blemish comprises a wrinkle.

31. Apparatus for treating a blemish having a furrow in a region of skin comprising:
a bracing body having a surface that is placed contiguous with or in proximity to the region of skin, said bracing body having at least one air passage through which air between the skin region and the surface can be aspirated; and
a vacuum pump coupled to the bracing body for aspirating air through the air passage so as to flatten the blemish to the bracing body surface.

32. Apparatus according to claim 31, wherein the bracing body comprises:
a thin dielectric layer bonded to a conductor, wherein the bracing body surface is a surface of the dielectric layer not bonded to the conductor; and
a power supply that applies a voltage difference between the conductor and the skin.

33. Apparatus according to claim 31 and comprising a deformable foil having an adhesive layer for bonding the foil to the region of skin and wherein when air is aspirated through the bracing body, the foil is flattened to the bracing body surface.

34. Apparatus according to claim 31 and comprising:
a foil having a dielectric layer bonded to a conducting layer, which foil is deformable so that the dielectric layer contacts skin in the furrow; and
a power supply that generates a potential difference between the conducting layer and the skin so that electrostatic forces adhere the foil to the skin in the furrow and wherein when air is aspirated through the bracing body, the foil is flattened to the bracing body surface.

35. Apparatus according to claim 33 wherein the bracing body and the foil are formed from a magnetized material and wherein once the foil is flattened to the bracing body surface, magnetic forces maintain the foil in contact with the bracing body surface.

36. Apparatus according to claim 31 wherein the bracing body is formed from a porous material and wherein the at least one air passage comprises air passages formed by pores in the material.

37. Apparatus according to claim 31 wherein the at least one air passage of the bracing body has particles on surfaces thereof that absorb a gas released through the skin.

38. Apparatus for treating a blemish having a farrow in a region of skin comprising:
a foil comprising magnetic material deformable so as to contact skin in the furrow, which foil, when in contact with skin, adheres to the skin through the agency of an adhesive or electrostatic forces;
a bracing body positioned in proximity to the foil; and
a magnetic element that attracts the foil and flattens the foil to the bracing body surface.

39. Apparatus according to claim 38 wherein the bracing comprises the magnetic element.

40. Apparatus for treating a skin blemish having a furrow comprising:
a bracing body having a surface;
an elastic foil having edges attached to the bracing body surface and a surface covered with an adhesive that faces away from the bracing body surface; and
an air passage that communicates with a space between the foil and the bracing body surface, wherein when air is forced through the air passage, the foil distends away from the surface and adheres to skin in the furrow and when air is subsequently aspirated through the passage the foil substantially recovers its shape flattening to the bracing body surface and flattening thereby the blemish.

41. Apparatus according to claim 40 and comprising a pump that forces air through the air passage.

42. Apparatus according to claim 40 wherein the bracing body is a thin rectangular ribbon shaped body.

43. Apparatus according to claim 31 wherein the bracing body surface is formed so that the contour of the surface substantially matches the contour of the region of skin exclusive of contour features resulting from the blemishes in the skin.

44. A method for treating blemishes having furrows on a region of a person's skin comprising:
heating the skin so that bent collagen fibers in the furrows become plastic;
applying forces to the heated collagen fibers that tend to straighten the fibers; and
cooling the collagen fibers while the forces are applied so that the collagen fibers retain a memory of their straightened configuration.

45. Apparatus according to claim 31 wherein a blemish comprises a wrinkle.

46. A method for treating blemishes on a region of person's skin comprising:
placing on the region of skin an elastically stretchable, multi-layered body comprising a conducting layer coated with a thin dielectric layer so that the surface of the dielectric layer is contiguous with the skin; and
repeatedly stretching and relaxing the layered body while the surface of the dielectric layer is in contact with the region of skin.

47. A method according to claim 44 wherein a blemish comprises a wrinkle.
